(19) United States
(12) Patent Application Publication
(10) Pub. No.: US 2025/0167271 A1
(43) Pub. Date: May 22, 2025

(54) Sodium Formate Hydrogen Extraction System Operation And Production Of Hydrogen And Methanol

(71) Applicant: NuScale Power, LLC, Corvallis, OR (US)

(72) Inventors: Francis Y. Tsang, Bellingham, WA (US); José N. Reyes, JR., Corvallis, OR (US); Luis DePavia, Corvallis, OR (US)

(73) Assignee: NuScale Power, LLC, Corvallis, OR (US)

(21) Appl. No.: 18/941,525

(22) Filed: Nov. 8, 2024

Related U.S. Application Data
(60) Provisional application No. 63/602,227, filed on Nov. 22, 2023.

(51) Int. Cl. H01M 8/0606; B01D 53/047; C01B 3/22; C01B 3/56; C01D 1/04; C07C 29/15; C07C 51/41; C25B 1/16; F01D 15/10; H01M 8/04082

(52) U.S. Cl. CPC H01M 8/0606; B01D 53/047; C01B 3/22; C01B 3/56; C01D 1/04; C07C 29/15; C07C 51/41; C07C 51/412; C25B 1/16; F01D 15/10; H01M 8/04201; B01D 2256/16; B01D 2257/502; B01D 2257/504; C01B 2203/0266; C01B 2203/043; C01B 2203/047; C01B 2203/0475; C01B 2203/061; C01B 2203/066; C01B 2203/1211; C01B 2203/84; F05D 2220/76; H01M 2250/10

(57) ABSTRACT

An integrated energy system comprising a power plant including at least one nuclear reactor and electrical power generation system, the at least one nuclear reactor being configured to generate steam, and the electrical power generation system being configured to generate electricity, a desalination system configured to receive at least a portion of the electricity and steam to produce brine, an electrolysis process configured to process the brine into Sodium Hydroxide (NaOH), a Sodium Formate (HCOONa) production process configured to receive the Sodium Hydroxide (NaOH) to produce Sodium Formate (HCOONa), a Hydrogen (H₂) extraction reactor configured to receive the Sodium Formate (HCOONa) and produce Hydrogen (H₂), and a fuel cell configured to receive the Hydrogen (H₂).

### CROSS-REFERENCE TO RELATED APPLICATIONS

This application claims the benefit of U.S. Provisional Patent Application No. 63/602,227 filed Nov. 22, 2023 and titled “SODIUM FORMATE HYDROGEN EXTRACTION SYSTEM OPERATION AND PRODUCTION OF HYDROGEN AND METHANOL,” which is incorporated herein by reference in its entirety.

### BACKGROUND

Modern society depends on a steady supply of electrical energy from a power grid whenever electrical energy is needed. Accordingly, a power grid requires a dependable source of electrical energy from energy producers in order to provide consistent electrical energy to consumers, whenever electrical energy is needed. Depending on the geographic location, population, and other factors, demand for electrical energy may be relatively high at certain times of the day and relatively lower at other times. For example, highly populated areas with hot climates may experience a large increase in energy demand in the evenings caused by a large number of consumers getting home from work and turning on air conditioning units. Similarly, the same highly populated areas with hot climates may see large decreases in energy demand in the evenings caused by the large number of consumers turning off air conditioners and going to bed. The times of high energy demand, or “peak times,” and times of low energy demand, or “off-peak times,” may be anticipated and planned for. As a result of the fluctuations between peak times and off-peak times, energy providers (e.g., nuclear, solar, natural gas, fossil fuel, etc.) may experience similar fluctuations in the demand for energy production (i.e., high energy production demand during peak times is greater than during off-peak times).

Hydrogen (H₂) is an energy carrier and one of the most important materials to the industrial world. In 2020, roughly 88 million metric tons of Hydrogen (H₂) were produced globally. More than 95% of produced Hydrogen (H₂) is generated through fossil fuels by: (1) steam-methane reforming of natural gas; (2) oxidation of hydrocarbons; (3) coal gasification; and/or (4) by biomass gasification. The prescribed processes generate very large Carbon Dioxide (CO₂) footprints that have been identified as a major source of greenhouse gases that contributes to climate change and global warming. Recently, electrolysis of water to produce Hydrogen (H₂) is becoming an important and integral part of the processes to reduce the greenhouse gas footprint of Hydrogen (H₂) production.

An energy imbalance market (“EIM”) is a means of supplying energy when and where it is needed to balance fluctuations in energy demand (i.e., peak times vs. off-peak times) and subsequent fluctuations in energy production demand (i.e., energy production demand during peak times vs. energy production demand during off-peak times). To increase productivity and efficiency for energy producers experiencing fluctuating energy production demand, new processes, systems, and methods are needed. Energy from power generation plants, such as, for example, nuclear reactors and/or renewable sources, can be diverted to produce Hydrogen (H₂) as an energy carrier for short-term storage during off-peak hours to support the EIM.

### BRIEF DESCRIPTION OF THE DRAWINGS

FIG. 1 schematically illustrates a representation of an integrated energy system 100 that includes a small modular nuclear reactor (SMR) system integrated with a Hydrogen (H₂) production system and a reversible solid oxide fuel cell (RSOFC).

FIG. 2 illustrates a steady-state Hydrogen (H₂) production process utilizing Sodium Formate (HCOONa) and a reverse solid oxide fuel cell (RSOFC).

FIG. 3 illustrates a steady-state Hydrogen (H₂) production process utilizing Sodium Formate (HCOONa) and a RSOFC.

FIG. 4 illustrates a steady-state Hydrogen (H₂) production process utilizing Sodium Formate (HCOONa) and a RSOFC.

FIG. 5 illustrates in-situ and on-demand Hydrogen (H₂) production system to support emergency and limited energy imbalance market (EIM) using Sodium Formate (HCOONa).

FIG. 6 schematically illustrates a representative schematic diagram of a Hydrogen (H₂) extraction reactor during steady state operations.

FIG. 7A schematically illustrates a representative schematic diagram of a Hydrogen (H₂) extraction reactor during steady state operations for the thermal decomposition of Sodium Formate (HCOONa) (i.e., dry process).

FIG. 7B schematically illustrates a representative schematic diagram of a Hydrogen (H₂) extraction reactor during steady state operations for the hydrothermal decomposition of Sodium Formate (HCOONa) (i.e., wet process).

FIG. 8 illustrates a Hydrogen (H₂) production process using multiple Sodium Formate (HCOONa) production systems simultaneously.

FIG. 9 illustrates an integrated energy system (IES) configured to capture atmospheric Carbon Dioxide (CO₂) to produce Carbon Dioxide (CO₂) and Carbon Monoxide (CO) for use in Methanol (CH₃OH) production.

FIG. 10 illustrates a flow diagram of an example process associated with utilizing a Hydrogen (H₂) extraction reactor and a solid oxide electrolysis cell (SOEC) for the simultaneous production of Sodium Oxalate ((COO)₂Na₂) and Formaldehyde (CH₂O).

FIG. 11 illustrates a flowchart describing an example process for utilizing a power plant system to produce Hydrogen (H₂) for use to produce electricity as necessary to support an EIM.

FIG. 12 is a partially schematic, partially cross-sectional view of a nuclear reactor system configured in accordance with embodiments of the present technology.

FIG. 13 is a partial schematic, partial cross-sectional view of a nuclear reactor system configured in accordance with additional embodiments of the present technology.

FIG. 14 is a schematic view of a nuclear power plant system including multiple nuclear reactors in accordance with embodiments of the present technology.

### DETAILED DESCRIPTION

The Detailed Description is set forth with reference to the accompanying figures. In the figures, the left-most digit(s) of a reference number identifies the figure in which the reference number first appears. The use of the same reference numbers in different figures indicates similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity.

### Overview

This disclosure is directed to an integrated SMR system that can be emplaced as a baseload energy generator to produce electricity for the power grid during periods where energy production demand is high (“peak times”). During the period when the demand for electricity is low or below the typical baseload supply (“off-peak times”), then some of the reactor systems (e.g., SMRs) can be utilized to provide electricity and steam to high-temperature steam electrolysis cell (HTSE) stacks to produce Hydrogen (H₂) and Oxygen (O₂) for storage. The stored Hydrogen (H₂) and Oxygen (O₂) may then be used to feed into an electrochemical device configured to operate in both an electrolysis mode (i.e., to produce Hydrogen (H₂)) and a fuel cell mode (i.e., to produce electricity) (e.g., a reversible solid oxide electrolysis cell (RSOEC), a reversible solid oxide fuel cell (RSOFC), solid oxide electrolysis cell (SOEC) stack, etc.) to generate electricity to support an EIM process. In some instances, the EIM time slot may typically be defined between 6:00 p.m. to 10:00 p.m. (about a 4-hour period).

In embodiments, the SMR system of the present disclosure can be a permanent or temporary installation built at or near (e.g., roughly 1 km from) the location of an industrial process facility or can be a mobile or partially mobile system that is moved to and assembled at or near the location of the industrial process facility. More generally, the SMR system can be local (e.g., positioned at or near) to the industrial processes/operations it supports. For example, the SMR system can be located within 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the SMR system is configured to supply a portion of electricity to a power grid.

Two pathways for Hydrogen (H₂) production with Sodium Formate (HCOONa) are shown below:

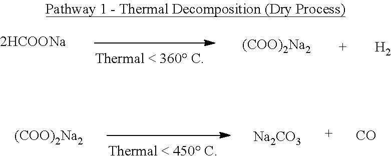

On heating, starting at about 250° C. up to about 450° C., Sodium Formate (HCOONa) decomposes to form Sodium Carbonate (Na₂CO₃), Hydrogen (H₂) gas, and Carbon Monoxide (CO) gas.

### Pathway 2—Hydrothermal Decomposition (Utilizing Super-Heated Steam)

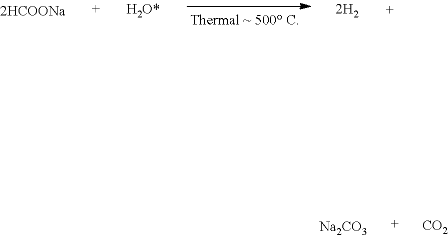

* super-heated steam (e.g., process steam) from the SMR system
   When super-heated steam (e.g., steam >500° C.) from a nuclear reactor system (e.g., an SMR system) is allowed to be injected into the system, the super-heated steam will react with Sodium Formate (HCOONa) and Hydrogen (H₂) is released and Carbon Dioxide (CO₂) is formed.

The present technology is directed to Hydrogen (H₂) and Methanol (CH₃OH) generation, such as from Sodium Formate (HCOONa), to support an energy imbalance market (EIM) and/or to provide a supplemental backup process to other Hydrogen (H₂) and Methanol (CH₃OH) production mechanisms.

### Illustrative Embodiments

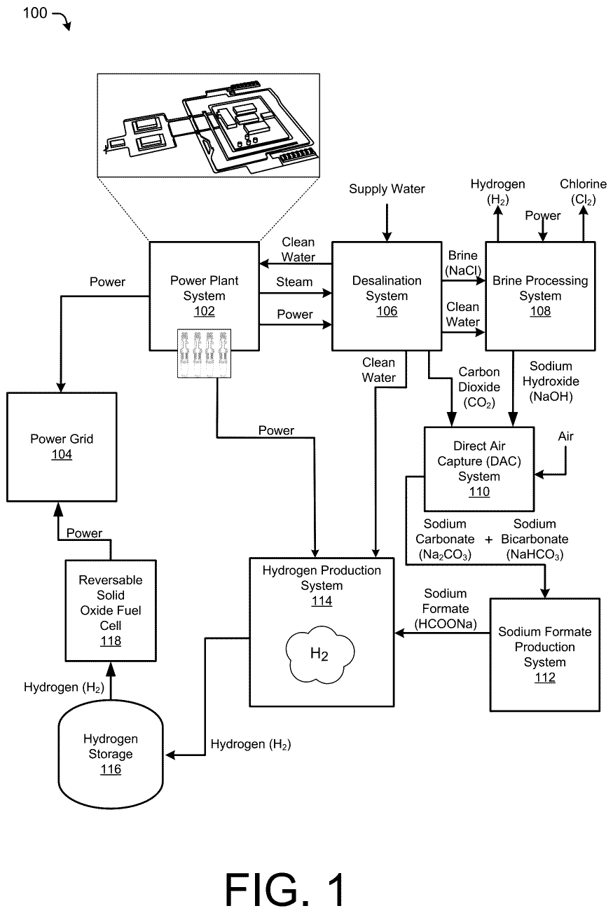

FIG. 1 schematically illustrates a representation of an integrated energy system 100 that includes a SMR system integrated with a Hydrogen (H₂) production system and an electrochemical device (e.g., a reversible solid oxide fuel cell (RSOFC)). The integrated energy system 100 may include a power plant system 102, a power grid 104, a desalination system 106, a brine processing system 108, a direct air capture (DAC) system 110, a Sodium Formate (HCOONa) production system 112, a Hydrogen (H₂) production system 114, a Hydrogen (H₂) storage 116, and a RSOFC 118.

In the illustrated embodiment, the power plant system 102 may be configured to provide electrical power directly to the power grid 104. In embodiments, the power plant system 102 may produce and deliver electrical power to the power grid 104 during peak times or anytime that there is a demand for energy production. For example, when consumer energy demand imposes a high energy demand on the power grid 104 (“peak times”), the power plant system 102 may be configured to produce and provide the energy necessary for the power grid 104 to meet the high consumer demand during peak times. In embodiments, the power plant system 102 may be configured to provide energy directly to the power grid 104 as required to meet energy demand due to factors other than increased energy demand during peak times (e.g., an energy producing plant that provides energy to the power grid 104 may be offline and unable to provide energy, which creates an increased energy production demand without an increased demand for consumer electrical power).

In the illustrated embodiment, the power plant system 102 may be configured to provide steam and power to the desalination system 106. In embodiments, the desalination system 106 may be configured to utilize the steam and power from the power plant system 102 to convert supply water into a concentrated NaCl solution (“brine”), clean water, and Carbon Dioxide (CO₂). In embodiments, the brine and the clean water may be directed into the brine processing system 108 and the Carbon Dioxide (CO₂) may be directed to the DAC system 110. The brine processing system 108 may be configured to convert brine into Sodium Hydroxide (NaOH), Hydrogen (H₂), and Chlorine (Cl₂). The Sodium Hydroxide (NaOH) may be directed from the brine processing system 108 to the DAC system 110. The DAC system 110 may be configured to convert the Sodium Hydroxide (NaOH) from the brine processing system 108, the Carbon Dioxide (CO₂) from the desalination system 106, and air into Sodium Carbonate (Na₂CO₃) and Sodium Bicarbonate (NaHCO₃). In embodiments, the Sodium Carbonate (Na₂CO₃) and Sodium Bicarbonate (NaHCO₃) from the DAC system 110 may be directed into the Sodium Formate (HCOONa) production system 112. Sodium Formate (HCOONa) may be produced in a large-scale inexpensively from Formic Acid (HCOOH) via carbonylation of Methanol (CH₃OH) followed by adding water to the resulting Methyl Formate (HCOOCH₃). Sodium Formate (HCOONa) may also be produced by neutralizing Formic Acid (HCOOH) with Sodium Hydroxide (NaOH).

In embodiments, the Sodium Formate (HCOONa) production system 112 may utilize the Sodium Carbonate (Na₂CO₃) and Sodium Bicarbonate (NaHCO₃) to generate Sodium Formate (HCOONa). The Sodium Formate (HCOONa) from the Sodium Formate (HCOONa) production system 112 may be directed into the Hydrogen (H₂) production system 114. In embodiments, the Hydrogen (H₂) production system 114 may be configured to receive clean water from the desalination system 106 and Sodium Formate (HCOONa) from the Sodium Formate (HCOONa) production system 112 and produce Hydrogen (H₂). In embodiments, the Hydrogen (H₂) may be directed from the Hydrogen (H₂) storage 116 to the RSOFC 118. The RSOFC 118 may be configured to utilize the Hydrogen (H₂) from the Hydrogen (H₂) storage 116 to produce electrical power. In embodiments, the electrical power produced by the RSOFC 118 may be directed to the power grid 104.

It is understood that the integrated energy system 100 may be configured such that the power plant system 102 may simultaneously produce electrical power directly to the power grid 104 and produce Hydrogen (H₂) via the desalination system 106, the brine processing system 108, the direct air capture (DAC) system 110, the Sodium Formate (HCOONa) production system 112, and the Hydrogen (H₂) production system 114. It is also understood that the integrated energy system 100 may be configured such that the power plant system 102 may simultaneously produce and provide electrical power directly to the power grid 104 while the RSOFC 118 is utilizing Hydrogen (H₂) from the Hydrogen (H₂) storage 116 to produce and provide electrical power directly to the power grid 104.

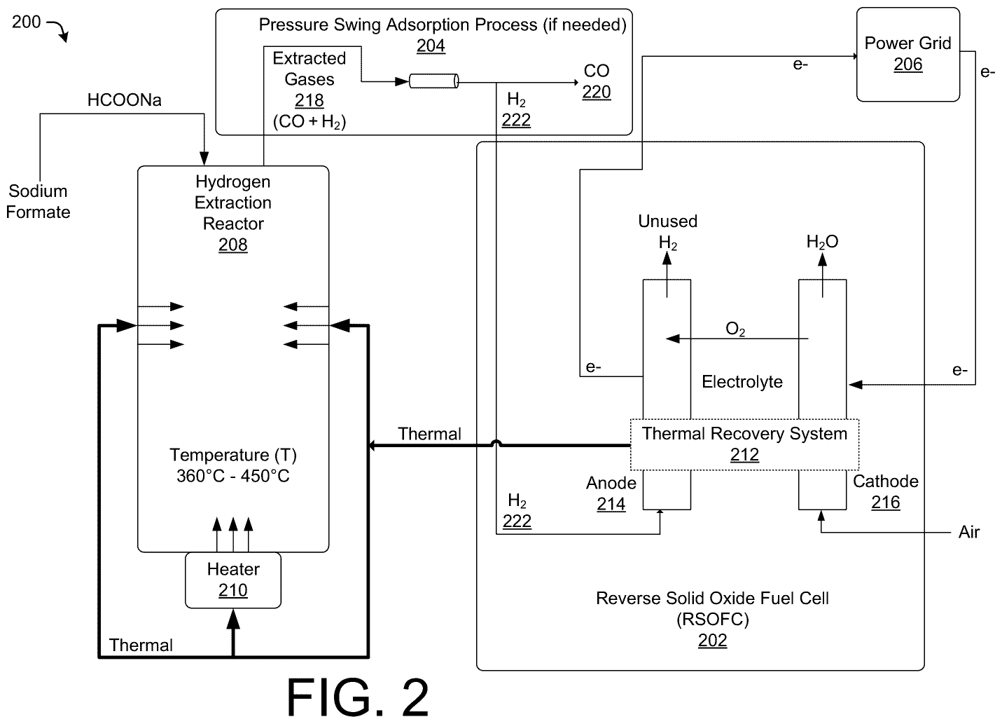

FIG. 2 illustrates a steady-state Hydrogen (H₂) production process 200 (“process 200”) utilizing Sodium Formate (HCOONa) and an electrochemical device (e.g., RSOFC 202). In embodiments, process 200 may include the RSOFC 202, the pressure swing adsorption process 204, the power grid 206, and the Hydrogen (H₂) extraction reactor 208. In embodiments, the Hydrogen (H₂) extraction reactor 208 may include the Hydrogen (H₂) extraction reactor heater 210. In embodiments, the RSOFC 202 may include the anode 214 and the cathode 216.

In embodiments, Sodium Formate (HCOONa) is fed into the Hydrogen (H₂) extraction reactor 208. The Hydrogen (H₂) extraction reactor heater and the RSOFC 202 may receive heat from the thermal recovery system 212 that is powered by the power grid 206 to keep the process 200 at operational temperatures. Excess Hydrogen (H₂) that is produced may be fed back into the anode 214 of the thermal recovery system 212. Hydrogen (H₂) may also be injected from an external source (e.g., a tanker truck, a storage tank, etc.) into the RSOFC 202 to generate electricity and reduce energy from the power grid 206. The thermal energy recovered by the thermal recovery system 212 within the RSOFC 202 may be used to maintain temperature in the Hydrogen (H₂) extraction reactor 208 and disconnect grid power (i.e., the thermal energy recovered from the thermal recovery system 212 may be sufficient to maintain the Hydrogen (H₂) extraction reactor 208 at operating temperatures, thus external power may not be required to energize the Hydrogen (H₂) extraction reactor heater 210). Sodium Formate (HCOONa) may be unloaded into the Hydrogen (H₂) extraction reactor 208 for steady-state Hydrogen (H₂) generation operations.

In embodiments, the Hydrogen (H₂) extraction reactor 208 may receive Sodium Formate (HCOONa). It is understood that the Hydrogen (H₂) extraction reactor 208 may receive Sodium Formate (HCOONa) in a solid-state form or as a powder. In the illustrated embodiment, the Hydrogen (H₂) extraction reactor 208 may be maintained with an internal temperature ranging from 360° C. to 450° C. In some embodiments, the Hydrogen (H₂) extraction reactor heater 210 may utilize electricity from the power grid 206 to maintain the Hydrogen (H₂) extraction reactor internal temperature. In some embodiments, the Hydrogen (H₂) extraction reactor heater 210 may utilize thermal energy recovered from the thermal recovery system 212 to maintain the Hydrogen (H₂) extraction reactor internal temperature.

During its operation, the Hydrogen (H₂) extraction reactor 208 may process Sodium Formate (HCOONa) to produce extracted gases 218 (e.g., Carbon Monoxide (CO), and Hydrogen (H₂)). It is understood that the extracted gases 218 may be a mixture of gases with varied concentrations. The extracted gases 218 may be directed to the pressure swing adsorption process 204 to be separated into separate gases (i.e., a mixture of Hydrogen (H₂) and Carbon Monoxide (CO) may be separated by the pressure swing adsorption process 204).

In embodiments, the Hydrogen (H₂) 222 may be directed to the RSOFC 202. It is understood the process 200 may use a Hydrogen (H₂) fuel cell other than the type depicted within FIG. 2. In the illustrated embodiment, the Hydrogen (H₂) 222 may be directed to the anode 214 of the RSOFC 202 to be oxidized and separated into negatively charged hydrogen electrons and positively charged hydrogen ions. The negatively charged hydrogen electrons may be directed to the power grid 206 to generate electricity before being directed to the cathode 216. In embodiments, air (e.g., atmospheric air containing Oxygen (O₂)) may be directed into the cathode 216 of the RSOFC 202. The Oxygen (O₂) in the cathode 216 may combine with the negatively charged hydrogen electrons directed into in the cathode 216 from the power grid 206 and the positively charged hydrogen particles that traveled from the anode 214 to the cathode 216 via the electrolyte to generate water (H₂O). The oxidation of the Hydrogen (H₂) 222 and generation of water (H₂O) in the RSOFC 202 may produce heat. In embodiments, the thermal recovery system 212 may capture the heat generated by the RSOFC 202 and direct the heat to Hydrogen (H₂) extraction reactor 208.

It is understood that the thermal recovery system 212 may be a system utilizing a thermal fluid to transfer heat, a system utilizing a Stirling engine with electrical component to power a heater, or any other system suitable to recover and reuse the heat generated by the RSOFC 202.

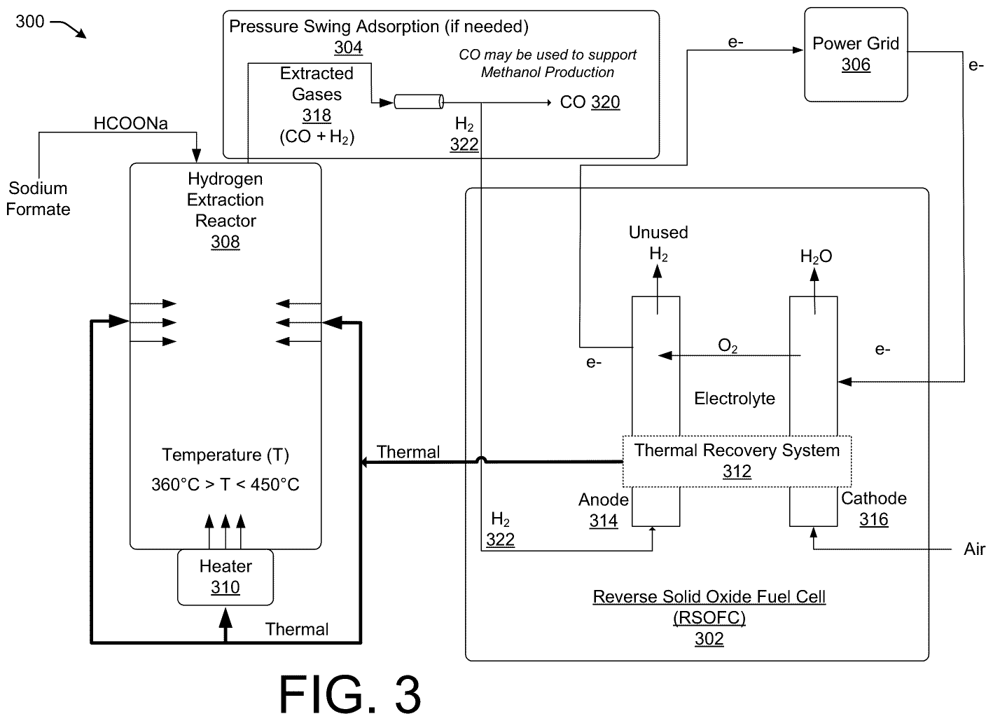

FIG. 3 illustrates a steady-state Hydrogen (H₂) production process 300 (“process 300”) utilizing Sodium Formate (HCOONa) and an electrochemical device (e.g., RSOFC 302). In embodiments, process 300 may include the RSOFC 302, the pressure swing adsorption process 304, the power grid 306, and the Hydrogen (H₂) extraction reactor 308. In embodiments, the Hydrogen (H₂) extraction reactor 308 may include the Hydrogen (H₂) extraction reactor heater 310. In embodiments, the RSOFC 302 may include the thermal recovery system 312, the anode 314, and the cathode 316.

In embodiments, Sodium Formate (HCOONa) is fed into the Hydrogen (H₂) extraction reactor 308. The Hydrogen (H₂) extraction reactor heater and the RSOFC 302 may receive heat from the thermal recovery system 312 that is powered by the power grid 306 to keep the process 300 at operational temperatures. Excess Hydrogen (H₂) that is produced may be fed back into the anode 314 of the thermal recovery system 312. Hydrogen (H₂) may also be injected from an external source (e.g., a tanker truck, a storage tank, etc.) into the RSOFC 302 to generate electricity and reduce energy from the power grid 306. The thermal energy recovered by the thermal recovery system 312 within the RSOFC 302 may be used to maintain temperature in the Hydrogen (H₂) extraction reactor 308 and disconnect grid power (i.e., the thermal energy recovered from the thermal recovery system 312 may be sufficient to maintain the Hydrogen (H₂) extraction reactor 308 at operating temperatures, thus external power may not be required to energize the Hydrogen (H₂) extraction reactor heater 310). Sodium Formate (HCOONa) may be unloaded into the Hydrogen (H₂) extraction reactor 308 for steady-state Hydrogen (H₂) generation operations.

In embodiments, the Hydrogen (H₂) extraction reactor 308 may receive Sodium Formate (HCOONa). It is understood that the Hydrogen (H₂) extraction reactor 308 may receive Sodium Formate (HCOONa) in a solid-state form or as a powder. In the illustrated embodiment, the Hydrogen (H₂) extraction reactor 308 may be maintained with an internal temperature of >360° C. and <450° C. In some embodiments, the Hydrogen (H₂) extraction reactor heater 310 may utilize electricity from the power grid 306 to maintain the Hydrogen (H₂) extraction reactor internal temperature. In some embodiments, the Hydrogen (H₂) extraction reactor heater 310 may utilize thermal energy recovered from the thermal recovery system 312 to maintain the Hydrogen (H₂) extraction reactor internal temperature.

During its operation, the Hydrogen (H₂) extraction reactor 308 may process Sodium Formate (HCOONa) to produce extracted gases 318 (e.g., Carbon Monoxide (CO) and Hydrogen (H₂)). It is understood that the extracted gases 318 may be a mixture of gases with varied concentrations. The extracted gases 318 may be directed to the pressure swing adsorption process 304 to be separated into separate gases (i.e., a mixture of Hydrogen (H₂) and Carbon Monoxide (CO) may be separated by the pressure swing adsorption process 304).

In embodiments, the Carbon Monoxide (CO) 320 may be used for Methanol (CH₃OH) production. In embodiments, the Hydrogen (H₂) 322 may be directed to the RSOFC 302. It is understood the process 300 may use a Hydrogen (H₂) fuel cell other than the type depicted within FIG. 3. In the illustrated embodiment, the Hydrogen (H₂) 322 may be directed to the anode 314 of the RSOFC 302 to be oxidized and separated into negatively charged hydrogen electrons and positively charged hydrogen ions. The negatively charged hydrogen electrons may be directed to the power grid 306 to generate electricity before being directed to the cathode 316. In embodiments, air (e.g., atmospheric air containing Oxygen (O₂)) may be directed into the cathode 316 of the RSOFC 302. The Oxygen (O₂) in the cathode 316 may combine with the negatively charged hydrogen electrons directed into in the cathode 316 from the power grid 306 and the positively charged hydrogen particles that traveled from the anode 314 to the cathode 316 via the electrolyte to generate water (H₂O). The oxidation of the Hydrogen (H₂) 322 and generation of water (H₂O) in the RSOFC 302 may produce heat. In embodiments, the thermal recovery system 312 may capture the heat generated by the RSOFC 302 and direct the heat to Hydrogen (H₂) extraction reactor 308.

It is understood that the thermal recovery system 312 may utilize a thermal fluid to transfer heat, a Stirling engine with an electrical component to power a heater, or other system components suitable to recover and reuse the heat generated by the RSOFC 302.

For example, the Hydrogen (H₂) extraction reactor heater and the RSOFC may receive heat from the thermal recovery system that is powered by the power grid to keep the system at operational temperatures. Excess Hydrogen (H₂) that is produced may be fed back into the anode of the thermal recovery system. The recovered thermal from the RSOFC may be used to maintain temperature in the Hydrogen (H₂) extraction reactor and disconnect grid power. Pressure swing absorption may also be used. Carbon Monoxide (CO) may be used to support Methanol (CH₃OH) production.

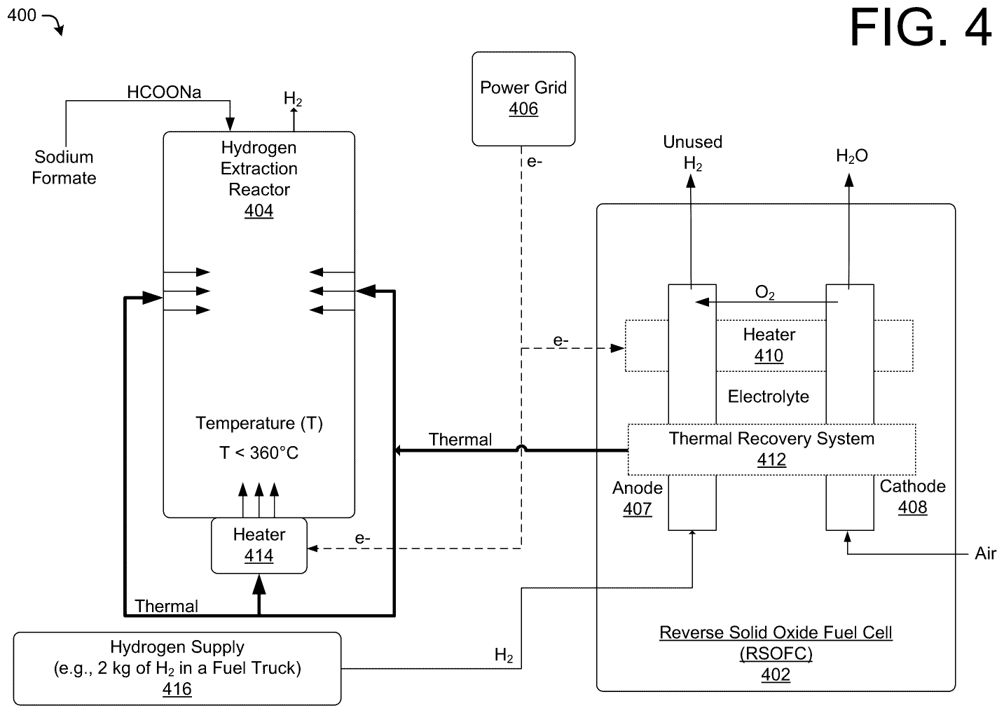

FIG. 4 illustrates a steady-state Hydrogen (H₂) production process 400 (“process 400”) utilizing Sodium Formate (HCOONa) and an electrochemical device (e.g., RSOFC 402). In embodiments, the process 400 may include the RSOFC 402, the Hydrogen (H₂) extraction reactor 404, and the power grid 406. In embodiment, the RSOFC 402 may include the anode 407, the cathode 408, the heater 410, and the thermal recovery system 412. In embodiments, the Hydrogen (H₂) extraction reactor 404 may include the Hydrogen (H₂) extraction reactor heater 414. In embodiments, the anode 407 may be configured to receive Hydrogen (H₂) from the Hydrogen (H₂) supply 416. It is understood that the Hydrogen (H₂) supply 416 may include a Hydrogen (H₂) production system, a permanent Hydrogen (H₂) storage tank, and/or a temporary Hydrogen (H₂) storage tank (e.g., a Hydrogen (H₂) fuel truck, portable tanks(s), etc.). In an embodiment, the RSOFC 402 may only need approximately 2 kg of Hydrogen (H₂) from an external source during startup. It is understood that in embodiments, external Hydrogen (H₂) may only be required for the RSOFC 402 during startup, and that after startup, the RSOFC 402 may receive necessary Hydrogen (H₂) from the Hydrogen (H₂) extraction reactor 404.

In embodiments, the power grid 406 may provide electrical power to the heater 410 and the Hydrogen (H₂) extraction reactor heater 414. It is understood that the heater 410 and the Hydrogen (H₂) extraction reactor heater 414 may only require the use of electrical power from the power grid 406 during startup. In some embodiments, electrical power may only be required for the heater 410 during startup, and that after startup, the RSOFC 402 may no longer require the use of the heater 410. For example, in embodiments, the RSOFC 402 may use the heater 410 during startup because the normal steady-state operation of the RSOFC 402 may generate the heat necessary to sustain the operation of the RSOFC 402.

In some embodiments, electrical power may only be required for the Hydrogen (H₂) extraction reactor heater 414 during startup, and that after startup, the Hydrogen (H₂) extraction reactor 404 may no longer require the use of the Hydrogen (H₂) extraction reactor heater 414. For example, in embodiments, the Hydrogen (H₂) extraction reactor heater 414 may use electrical energy to provide the heat necessary for the operation of the Hydrogen (H₂) extraction reactor 404 during startup. In embodiment, the normal steady-state operation of the RSOFC 402 may generate heat, which may be recovered by the thermal recovery system 412 and transferred to the Hydrogen (H₂) extraction reactor 404. During normal steady-state operation, the heat recovered by the thermal recovery system 412 may be adequate for operation of the Hydrogen (H₂) extraction reactor 404.

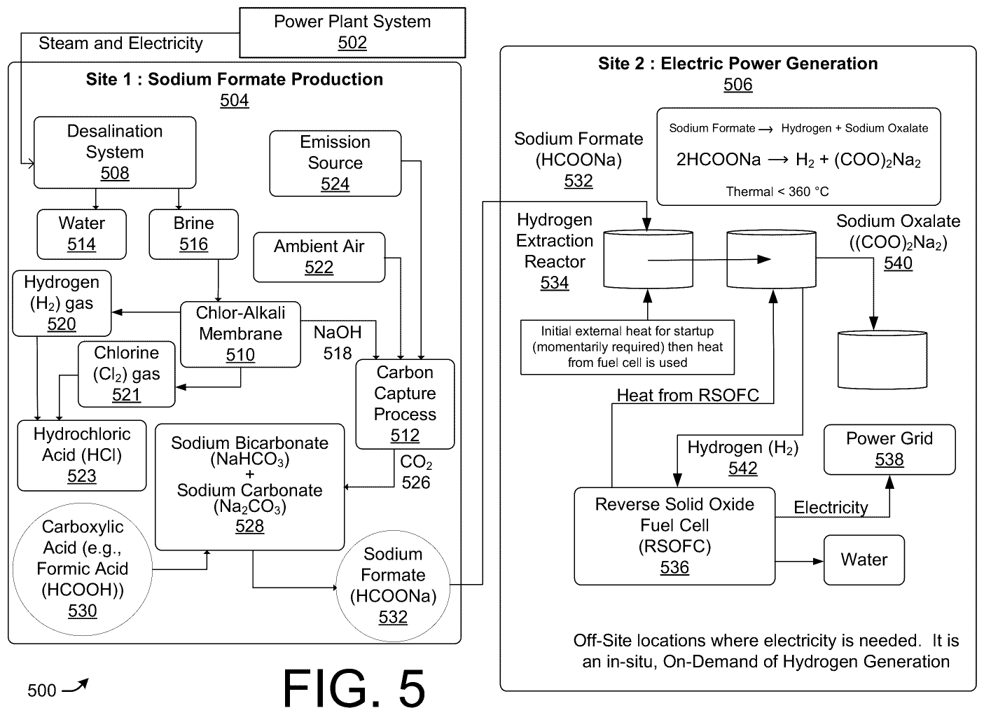

FIG. 5 illustrates in-situ and on-demand Hydrogen (H₂) production system (“system 500”) to support emergency and limited energy imbalance market (EIM) using Sodium Formate (HCOONa). In embodiments, the system 500 may include a power plant system 502, a first site 504, and a second site 506. The first site 504 may be used for Sodium Formate (HCOONa) production. The second site 506 may be an off-site location (i.e., located wherever electricity is needed that may not be in proximity to the power plant system 500). The second site 506 may provide in-situ on-demand Hydrogen (H₂) generation. In embodiments, the power plant system 500 may include the SMR system.

In embodiments, the first site 504 may be used for Sodium Formate (HCOONa) production. The first site 504 may include the desalination system 508, the chlor-alkali membrane 510, and the carbon capture process 512. It is understood the chlor-alkali membrane 510 may include any type of electrolysis system and/or process configured to process brine into Sodium Hydroxide (NaOH). At the first site 504, the power plant system 502 may supply steam and electricity to the desalination system 508. The desalination system 508 may produce water 514 and brine 516 (i.e., a concentrated Sodium Chloride (NaCl) solution). The brine 516 may be directed into the chlor-alkali membrane 510. The chlor-alkali membrane 510 may be configured to receive the brine 516 and generate Sodium Hydroxide (NaOH) 518, Hydrogen (H₂) gas 520, and Chlorine (Cl₂) gas 521 via electrolysis. In embodiments, the Hydrogen (H₂) gas 520 and the Chlorine (Cl₂) gas 521 may be combined to form Hydrochloric Acid (HCl) 523. In embodiments, the production of Hydrochloric Acid (HCl) may be represented by the equation below:

H₂+Cl₂→2HCl

In embodiments, the carbon capture process 512 may receive ambient air 522 (e.g., atmospheric air containing Carbon Dioxide (CO₂)) and/or an emission source 524 (e.g., gases containing Carbon Dioxide (CO₂) released as an emission from a process, machine, device, etc.) and produce Carbon Dioxide (CO₂) 526, which may be useful for industrial processes. In embodiments, the Sodium Hydroxide (NaOH) 518 may be combined with the Carbon Dioxide (CO₂) 526 to generate Sodium Bicarbonate (NaHCO₃) and Sodium Carbonate (Na₂CO₃) 528. In embodiments, a carboxylic acid (e.g., Formic Acid (HCOOH)) 530 may be reacted with the Sodium Bicarbonate (NaHCO₃) and Sodium Carbonate (Na₂CO₃) 528 to produce Sodium Formate (HCOONa) 532 that may be transported to the second site 506.

In embodiments, the second site 506 may include the Hydrogen (H₂) extraction reactor 534, an electrochemical device (e.g., reverse solid oxide fuel cell (RSOFC) 536), and the power grid 538. In embodiments, the Hydrogen (H₂) extraction reactor 534 may receive Sodium Formate (HCOONa) 532 to produce Sodium Oxalate ((COO)₂Na₂) 540 and Hydrogen (H₂) 542. In embodiments, the conversion of the Sodium Formate (HCOONa) 532 to Sodium Oxalate ((COO)₂Na₂) 540 and Hydrogen (H₂) 542 may be represented by the reaction below:

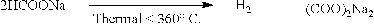

The Hydrogen (H₂) 542 may be directed to the RSOFC 536, which may be configured to convert the Hydrogen (H₂) 542 into electricity and water. It is understood that the Hydrogen (H₂) 542 may be directly directed to the RSOFC 536 and/or directed to a Hydrogen (H₂) tank (i.e., tanker truck, portable storage tank, permanently installed tank, etc.). The electricity produced by the RSOFC 536 may be directed to the power grid 538. In some embodiments, the RSOFC 536 may operate to produce electricity as needed to support an EIM. In embodiments, the RSOFC 536 may generate heat during operation which may be directed to the Hydrogen (H₂) extraction reactor 534.

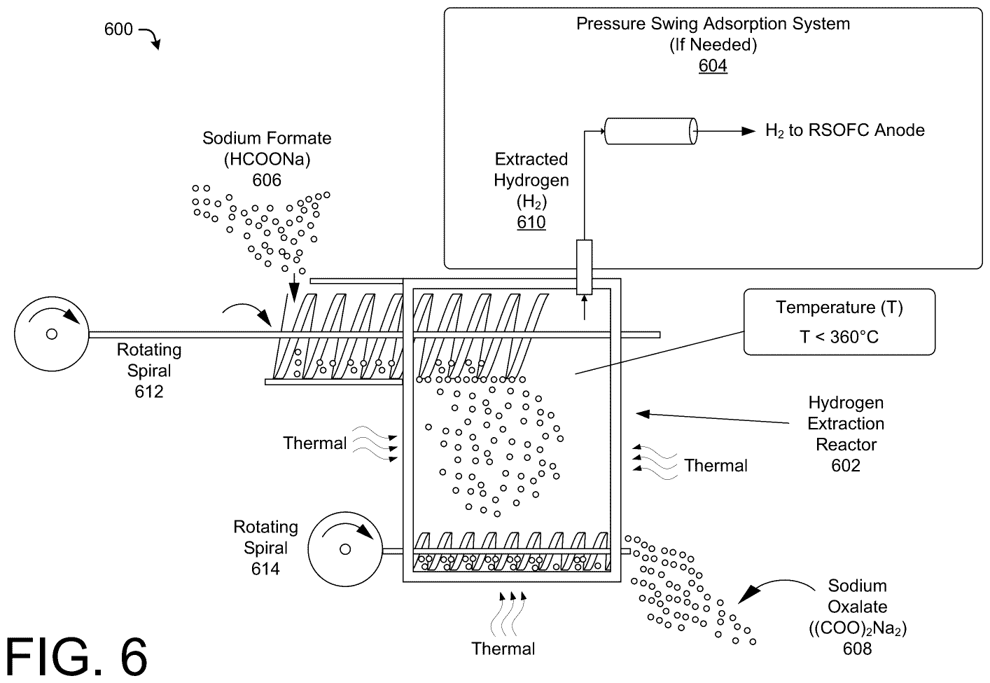

FIG. 6 schematically illustrates a representative schematic diagram of a Hydrogen (H₂) extraction reactor system 600 (“system 600”) during steady state operations. In embodiments, the system 600 may include the Hydrogen (H₂) extraction reactor 602 and a pressure swing adsorption system 604. The Hydrogen (H₂) extraction reactor 602 may be configured to receive Sodium Formate (HCOONa) 606 and produce Sodium Oxalate ((COO)₂Na₂) 608 and/or extracted Hydrogen (H₂) 610. The Sodium Formate (HCOONa) 606 may be directed, via the rotating spiral 612 (e.g., first rotating spiral), into an upper portion of the Hydrogen (H₂) extraction reactor 602. The Hydrogen (H₂) extraction reactor 602 may have an internal temperature <360° C.

In various cases, the rotating spiral 612 may be utilized to convert the Sodium Formate (HCOONa) 606 between particles of different sizes. For example, the rotating spiral 612 may be utilized to convert the Sodium Formate (HCOONa) 606 from relatively coarser (e.g., larger) particles to relatively finer (e.g., smaller) particles. The rotating spiral 612 may be utilized to assist in a conversion between the Sodium Formate (HCOONa) 606 to the Sodium Oxalate ((COO)₂Na₂) 608. The rotating spiral 612 may be utilized to maintain the temperature in the Hydrogen (H₂) extraction reactor 602 by providing a means to feed the Sodium Formate (HCOONa) 606 into the upper portion of the Hydrogen (H₂) extraction reactor 602 while minimizing the potential for heat loss. The rotating spiral 612 may be a metal rotating spiral (e.g., an auger), which may be located partially and/or fully in the first Hydrogen (H₂) extraction reactor 602. In embodiments, the rotating spiral 612 may be operated by a control system utilized to control any portion of the system 600 to rotate (e.g., spin) at one or more predetermined, and/or dynamically determined (e.g., in real-time), speeds during any operation of the system 600.

In an embodiment, the Sodium Formate (HCOONa) 606, may receive thermal energy as a result of the temperature inside the Hydrogen (H₂) extraction reactor 602. The internal temperature of the Hydrogen (H₂) extraction reactor 602 may cause the Sodium Formate (HCOONa) 606 to rapidly decompose into the extracted Hydrogen (H₂) 610 and the Sodium Oxalate ((COO)₂Na₂) 608. In the embodiment, the extracted Hydrogen (H₂) 610 may be produced instantaneously following the decomposition of the Sodium Formate (HCOONa) 606 into the Sodium Oxalate ((COO)₂Na₂) 608. In the embodiment, the resulting Sodium Oxalate ((COO)₂Na₂) 608 sinks to the bottom of the Hydrogen (H₂) extraction reactor 602 while still being thermally hot. The rotating spiral 614 (e.g., second rotating spiral) may transfer the thermally hot Sodium Oxalate ((COO)₂Na₂) 608 from the bottom of the Hydrogen (H₂) extraction reactor 602 to outside the Hydrogen (H₂) extraction reactor 602 for collection and/or additional industrial processing.

In embodiments, the pressure swing adsorption system 604 may be used to purify the extracted Hydrogen (H₂) 610 for future processing. In embodiments, the extracted Hydrogen (H₂) 610 may be directed into a portion of an electrochemical device (e.g., an RSOFC anode) and used for producing electricity.

For example, Sodium Formate (HCOONa) is fed into an extraction reactor chamber. In some embodiments, Sodium Formate (HCOONa) may be in a solid state and ground into fine powder via a rotating spiral such that the Sodium Formate (HCOONa) is disintegrated within the chamber. The recovered thermal maintains the temperature inside the extraction reactor chamber at <360° C. and Sodium Oxalate ((COO)₂Na₂) is produced. The reactor chamber may be coupled to a pressure swing absorption system to purify the Hydrogen (H₂) gas to be fed into the RSOFC anode.

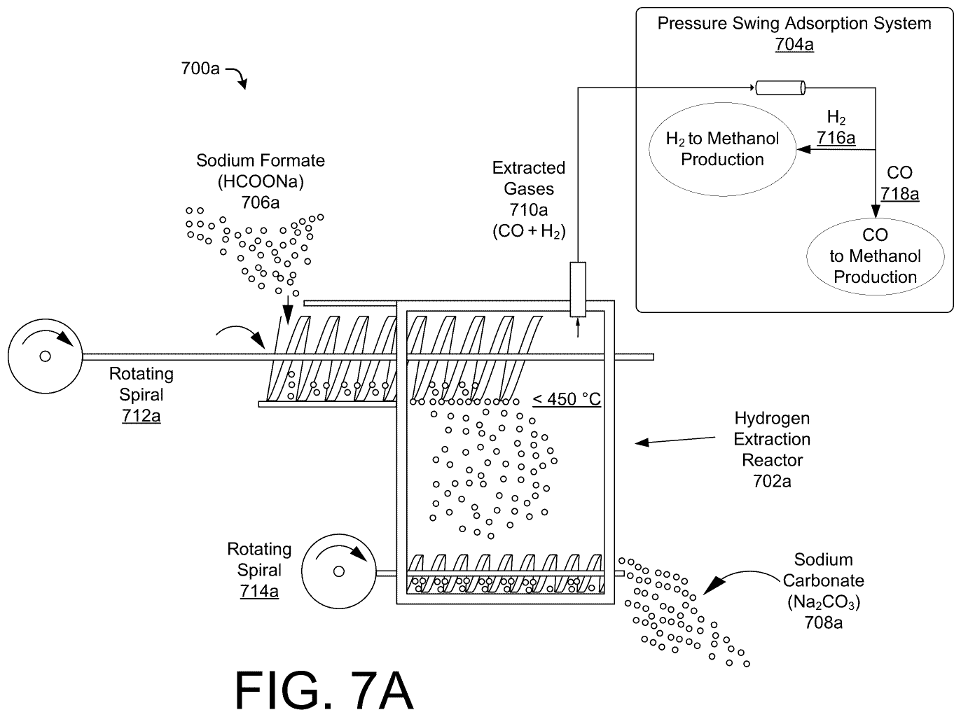

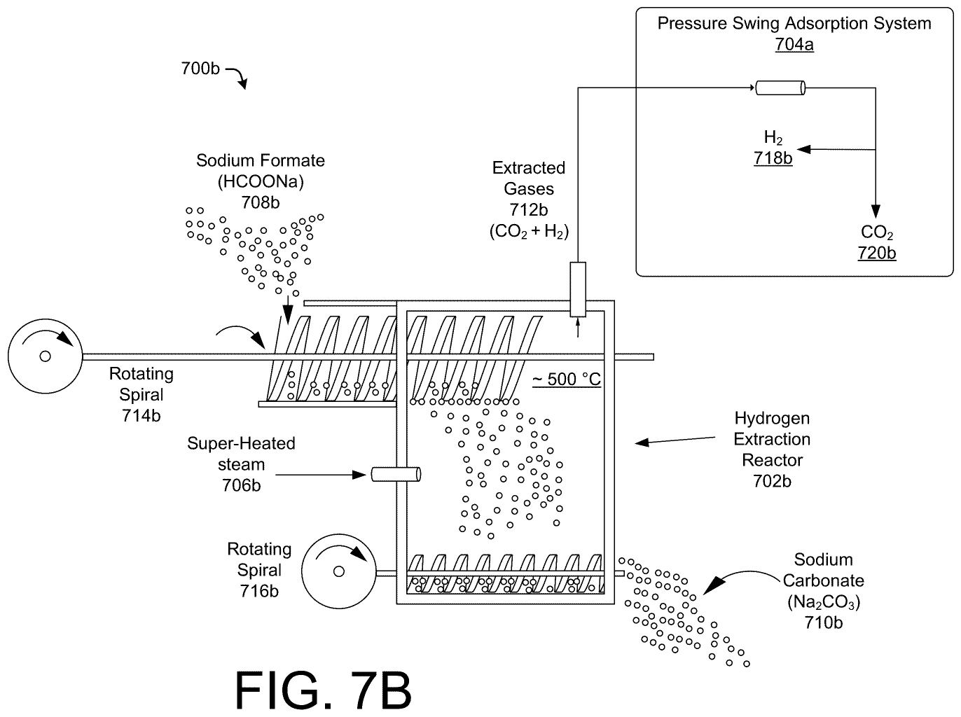

FIG. 7A schematically illustrates a representative schematic diagram of a Hydrogen (H₂) extraction reactor system 700a (“system 700a”) during steady state operations for the thermal decomposition of Sodium Formate (HCOONa) (i.e., dry process). In embodiments, the system 700a may include the Hydrogen (H₂) extraction reactor 702a and a pressure swing adsorption system 704a. The Hydrogen (H₂) extraction reactor 702a may be configured to receive Sodium Formate (HCOONa) 706a and produce Sodium Carbonate (Na₂CO₃) 708a and/or extracted gases 710a. The Sodium Formate (HCOONa) 706a may be directed, via the rotating spiral 712a (e.g., first rotating spiral), into an upper portion of the Hydrogen (H₂) extraction reactor 702a. The Hydrogen (H₂) extraction reactor 702a may have an internal temperature of <450° C.

In various cases, the rotating spiral 712a may be utilized to convert the Sodium Formate (HCOONa) 706a between particles of different sizes. For example, the rotating spiral 712a may be utilized to convert the Sodium Formate (HCOONa) 706a from relatively coarser (e.g., larger) particles to relatively finer (e.g., smaller) particles. The rotating spiral 712a may be utilized to assist in a conversion between the Sodium Formate (HCOONa) 706a to the Sodium Carbonate (Na₂CO₃) 708a. The rotating spiral 712a may be utilized to maintain the temperature in the Hydrogen (H₂) extraction reactor 702a by providing a means to feed the Sodium Formate (HCOONa) 706a into the upper portion of the Hydrogen (H₂) extraction reactor 702a while minimizing the potential for heat loss. The rotating spiral 712a may be a metal rotating spiral (e.g., an auger), which may be located partially and/or fully in the first Hydrogen (H₂) extraction reactor 702a. In embodiments, the rotating spiral 712a may be operated by a control system utilized to control any portion of the system 700a to rotate (e.g., spin) at one or more predetermined, and/or dynamically determined (e.g., in real-time), speeds during any operation of the system 700a.

In an embodiment, the Sodium Formate (HCOONa) 706a, may receive thermal energy as a result of the temperature inside the Hydrogen (H₂) extraction reactor 702a. The internal temperature of the Hydrogen (H₂) extraction reactor 702a may cause the Sodium Formate (HCOONa) 706a to rapidly decompose into the extracted gases 710a and the Sodium Carbonate (Na₂CO₃) 708a. In the embodiment, the extracted gases 710a may be produced instantaneously following the decomposition of the Sodium Formate (HCOONa) 706a into the Sodium Carbonate (Na₂CO₃) 708a. In the embodiment, the resulting Sodium Carbonate (Na₂CO₃) 708a sinks to the bottom of the Hydrogen (H₂) extraction reactor 702a while still being thermally hot. The rotating spiral 714a (e.g., second rotating spiral) may transfer the thermally hot Sodium Carbonate (Na₂CO₃) 708a from the bottom of the Hydrogen (H₂) extraction reactor 702a (i.e., the lower portion of the Hydrogen (H₂) extraction reactor) to outside the Hydrogen (H₂) extraction reactor 702a for collection and/or additional industrial processing.

In embodiments, the extracted gases 710a may include a mixture of Carbon Monoxide (CO) and Hydrogen (H₂). The pressure swing adsorption system 704 may be used to separate the extracted gases 710a into Hydrogen (H₂) 716a and Carbon Monoxide (CO) 718a. In embodiments, the Hydrogen (H₂) 716a and the Carbon Monoxide (CO) 718a may be used for Methanol (CH₃OH) production.

Basic ingredients and infrastructures for Methanol (CH₃OH) production include Hydrogen (H₂) and Carbon Monoxide (CO), as demonstrated by the reaction below:

CO+2H₂↔CH₃OH

In various embodiments, Copper (Cu) and/or Zinc Oxide (ZnO) is used as a catalyst, at 200° C.-300° C. and 50-100 bar.

FIG. 7B schematically illustrates a representative schematic diagram of a Hydrogen (H₂) extraction reactor system 700b (“system 700b”) during steady state operations for the hydrothermal decomposition of Sodium Formate (HCOONa) (i.e., wet process). In embodiments, the system 700b may include the Hydrogen (H₂) extraction reactor 702b and a pressure swing adsorption system 704b. The Hydrogen (H₂) extraction reactor 702b may be configured to receive super-heated steam 706b and Sodium Formate (HCOONa) 708b and produce Sodium Carbonate (Na₂CO₃) 710b and/or extracted gases 712b. The Sodium Formate (HCOONa) 708b may be directed, via the rotating spiral 714b (e.g., first rotating spiral), into an upper portion of the Hydrogen (H₂) extraction reactor 702b. The Hydrogen (H₂) extraction reactor 702b may have an internal temperature of ˜500° C. It is understood that the super-heated steam 706b may be steam (e.g., process steam) from an SMR system.

In various cases, the rotating spiral 714b may be utilized to convert the Sodium Formate (HCOONa) 708b between particles of different sizes. For example, the rotating spiral 714b may be utilized to convert the Sodium Formate (HCOONa) 708b from relatively coarser (e.g., larger) particles to relatively finer (e.g., smaller) particles. The rotating spiral 714b may be utilized to assist in a conversion between the Sodium Formate (HCOONa) 708b to the Sodium Carbonate (Na₂CO₃) 710b. The rotating spiral 714b may be utilized to maintain the temperature in the Hydrogen (H₂) extraction reactor 702b by providing a means to feed the Sodium Formate (HCOONa) 708b into the upper portion of the Hydrogen (H₂) extraction reactor 702b while minimizing the potential for heat loss. The rotating spiral 714b may be a metal rotating spiral (e.g., an auger), which may be located partially and/or fully in the first Hydrogen (H₂) extraction reactor 702b. In embodiments, the rotating spiral 714b may be operated by a control system utilized to control any portion of the system 700b to rotate (e.g., spin) at one or more predetermined, and/or dynamically determined (e.g., in real-time), speeds during any operation of the system 700b.

In an embodiment, the Sodium Formate (HCOONa) 708b, may receive thermal energy as a result of the temperature inside the Hydrogen (H₂) extraction reactor 702b. The internal temperature of the Hydrogen (H₂) extraction reactor 702b may cause the Sodium Formate (HCOONa) 708b to rapidly decompose into the extracted gases 712b and the Sodium Carbonate (Na₂CO₃) 710b. In the embodiment, the extracted gases 712b may be produced instantaneously following the decomposition of the Sodium Formate (HCOONa) 708b into the Sodium Carbonate (Na₂CO₃) 710b. In the embodiment, the resulting Sodium Carbonate (Na₂CO₃) 710b sinks to the bottom of the Hydrogen (H₂) extraction reactor 702b while still being thermally hot. The rotating spiral 716b (e.g., second rotating spiral) may transfer the thermally hot Sodium Carbonate (Na₂CO₃) 710b from the bottom of the Hydrogen (H₂) extraction reactor 702b (i.e., the lower portion of the Hydrogen (H₂) extraction reactor) to outside the Hydrogen (H₂) extraction reactor 702b for collection and/or additional industrial processing.

In embodiments, the extracted gases 712b may include a mixture of Carbon Dioxide (CO₂) and Hydrogen (H₂). The pressure swing adsorption system 704b may be used to separate the extracted gases 712b into Hydrogen (H₂) 718a and Carbon Dioxide (CO₂) 720b. In embodiments, the Hydrogen (H₂) 718b and the Carbon Dioxide (CO₂) 720b may be used for future processing.

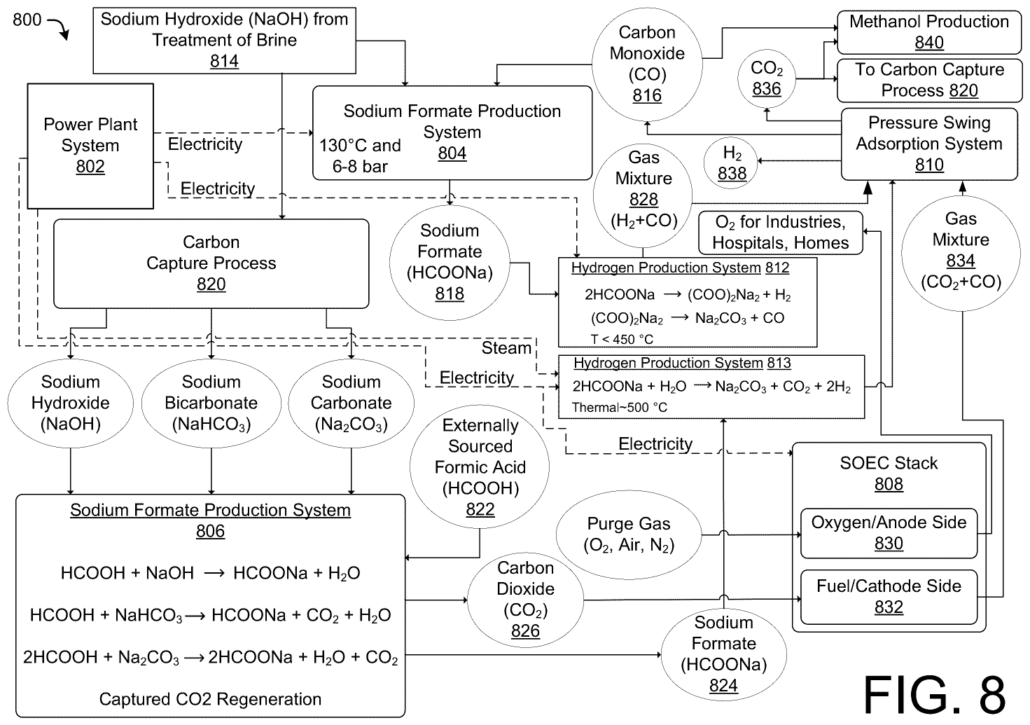

FIG. 8 illustrates Hydrogen (H₂) production process 800 (“process 800”) that uses multiple Sodium Formate (HCOONa) production systems simultaneously. The process 800 may include the power plant system 802, the Sodium Formate (HCOONa) production system 804 (e.g., first Sodium Formate (HCOONa) production system) and the Sodium Formate (HCOONa) production system 806 (e.g., second Sodium Formate (HCOONa) production system), an electrochemical device (e.g., solid oxide electrolysis cell (SOEC) stack 808), the pressure swing adsorption system 810, the Hydrogen (H₂) production system 812, and the Hydrogen (H₂) production system 813. In embodiments, the power plant system 802 may provide electricity to the Sodium Formate (HCOONa) production system 804, the Hydrogen (H₂) production system 812, the Hydrogen (H₂) production system 813, and the SOEC stack 808. In embodiments, the power plant system 802 may include an SMR system.

In embodiments, the Sodium Formate (HCOONa) production system 804 may be configured to receive Sodium Hydroxide (NaOH) 814 (e.g., first Sodium Hydroxide (NaOH)) that may be produced via treatment of brine (e.g., treatment of brine produced by a desalination system) and Carbon Monoxide (CO) 816 produced by the pressure swing adsorption system 810 to produce Sodium Formate (HCOONa) 818. In embodiments, the Sodium Formate (HCOONa) production system 804 may have operating parameters including 130° C. and a pressure range of 6-8 Bar. Production of Sodium Formate (HCOONa) 818 via the Sodium Formate (HCOONa) production system 804 may be represented by the following reaction:

NaOH (Dry Solid form)+CO→HCOONa

In embodiments, the Sodium Formate (HCOONa) 818 may be directed into the Hydrogen (H₂) production system 812.

In embodiments, the Sodium Formate (HCOONa) production system 806 may be configured to receive Sodium Hydroxide (NaOH) (e.g., second Sodium Hydroxide (NaOH)), Sodium Bicarbonate (NaHCO₃), and Sodium Carbonate (Na₂CO₃) from the carbon capture process 820, and externally sourced Formic Acid (HCOOH) 822 to produce Sodium Formate (HCOONa) 824 and Carbon Dioxide (CO₂) 826. The Sodium Formate (HCOONa) 824 may be directed to the Hydrogen (H₂) production system 813. In embodiments, the production of Sodium Formate (HCOONa) 824 via the Sodium Formate (HCOONa) production system 806 using Formic Acid (HCOOH) from an external supply and a solution of Sodium Hydroxide (NaOH), Sodium Bicarbonate (NaHCO₃), and Sodium Carbonate (Na₂CO₃) from the carbon capture process 820 (e.g., DAC unit) may be represented by the following reactions:

HCOOH+NaOH→HCOONa+H₂O

2HCOOH+Na₂CO₃→2HCOONa+H₂O+CO₂

HCOOH+NaHCO₃→HCOONa+H₂O+CO₂

In embodiments, the Hydrogen (H₂) production system 812 may have an operating temperature range of 250° C. and less than 450° C. to produce gas mixture 828. Production of gas mixture 828 via the Hydrogen (H₂) production system 812 may be represented by the following reactions:

2HCOONa→(COO)₂Na₂+H₂

(COO)₂Na₂→Na₂CO₃+CO

The gas mixture 828 may include Hydrogen (H₂) and Carbon Monoxide (CO), and the gas mixture 828 may be directed to the pressure swing adsorption system 810 for processing.

In embodiments, the Hydrogen (H₂) production system 813 may be configured to receive process steam from the power plant system 802. The Hydrogen (H₂) production system 813 may have an operating temperature of approximately 500° C. to produce a mixture of Carbon Dioxide (CO₂) and Hydrogen (H₂). Production of the Carbon Dioxide (CO₂) and the Hydrogen (H₂) via the Hydrogen (H₂) production system 813 may be represented by the following equation:

2HCOONa+H₂O (500° C. steam)→Na₂CO₃+2H₂+CO₂

The Carbon Dioxide (CO₂) and the Hydrogen (H₂) produced by the Hydrogen (H₂) production system 813 may be directed to the pressure swing adsorption system 810 for processing.

In embodiments, the SOEC stack 808 may include the Oxygen/anode side 830 and the fuel/cathode side 832. The Oxygen/anode side 830 may receive purge gas (e.g., Oxygen (O₂), atmospheric air, Nitrogen (N₂), etc.). The fuel/cathode side 832 may receive the Carbon Dioxide (CO₂) 826 produced by the Sodium Formate (HCOONa) production system 806. The SOEC stack 808 may produce Oxygen (O₂) for use in hospitals, homes, and other industries. The SOEC stack 808 may produce gas mixture 834. In embodiments, the gas mixture 834 may include Carbon Monoxide (CO) and Carbon Dioxide (CO₂). The gas mixture 834 may be directed to the pressure swing adsorption system 810.

In embodiments, the pressure swing adsorption system 810 may be configured to receive the gas mixture 834 from the SOEC stack 808, the gas mixture 828 from the Hydrogen (H₂) production system 812, and Carbon Dioxide (CO₂) and Hydrogen (H₂) from the Hydrogen (H₂) Production system 813. The pressure swing adsorption system 810 may produce the Carbon Monoxide (CO) 816, the Carbon Dioxide (CO₂) 836, and the Hydrogen (H₂) 838. The Carbon Monoxide (CO) 816 may be directed to the Sodium Formate (HCOONa) production system 804 to be used to produce Sodium Formate (HCOONa) and/or to the Methanol (CH₃OH) production system 840 to combine with Hydrogen (H₂) for the production of Methanol (CH₃OH). The Carbon Dioxide (CO₂) 836 may be directed to the carbon capture process 820 to be recaptured and reused, and/or the Carbon Dioxide (CO₂) 836 may be used for Methanol (CH₃OH) production by the Methanol (CH₃OH) production system 840. The Hydrogen (H₂) 838 may be used to produce electricity (e.g., directed to an RSOFC) to help manage an EIM, and/or collected for storage (e.g., permanent tank, portable tank, etc.).

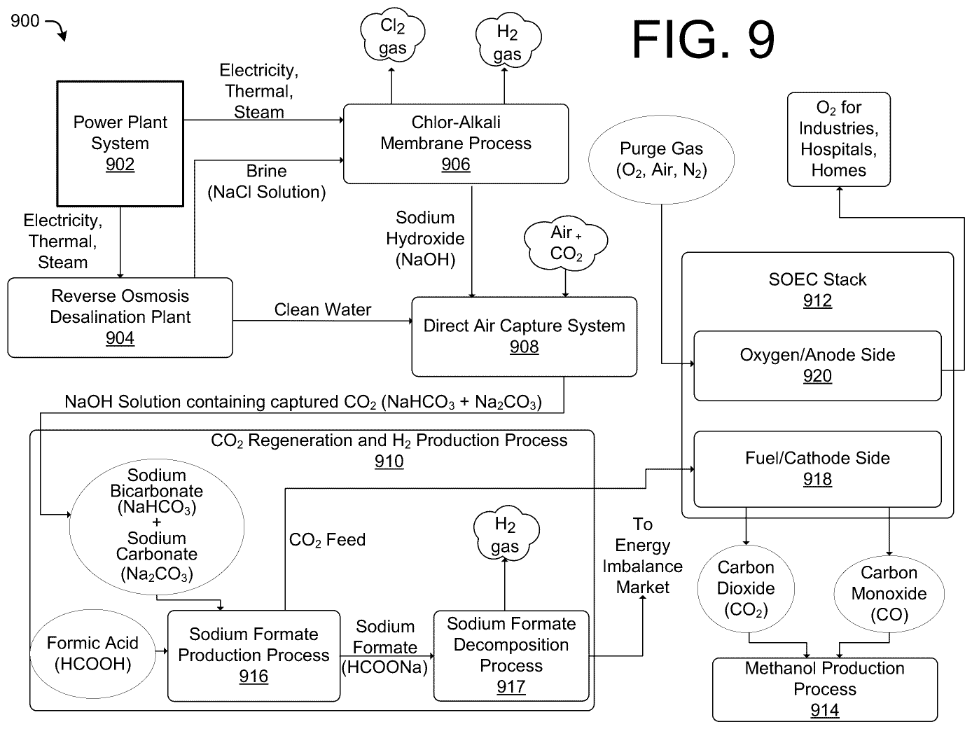

FIG. 9 illustrates an integrated energy system (IES) 900 configured to capture and produce Carbon Dioxide (CO₂) and Carbon Monoxide (CO) for use in Methanol (CH₃OH) production. In embodiments, the IES 900 may include the power plant system 902, the reverse osmosis desalination plant (“desalination plant”) 904, the chlor-alkali membrane process 906, the direct air capture (“DAC”) system 908, the CO₂ regeneration and H₂ production process 910, an electrochemical device (e.g., the SOEC stack 912), and the Methanol (CH₃OH) production process 914. In embodiments, the power plant system 902 may include a SMR system. The power plant system 902 may provide electricity, thermal, and steam to the desalination plant 904 and the chlor-alkali membrane process 906. The desalination plant 904 may provide clean water to the DAC system 908 and may provide brine (NaCl solution) to the chlor-alkali membrane process 906. The chlor-alkali membrane process 906 may produce Sodium Hydroxide (NaOH), Chlorine (Cl₂) gas, and Hydrogen (H₂) gas. The Sodium Hydroxide (NaOH) may be directed to the DAC system 908.

In embodiments, the DAC system 908 may be configured to receive clean water from the desalination plant 904, Sodium Hydroxide (NaOH) from the chlor-alkali membrane process 906, and air from the atmosphere (e.g., atmospheric Carbon Dioxide (CO₂)), and produce Sodium Carbonate (Na₂CO₃) and Sodium Bicarbonate (NaHCO₃) (e.g., an NaOH solution containing the captured Carbon Dioxide (CO₂)). The Sodium Carbonate (Na₂CO₃) and the Sodium Bicarbonate (NaHCO₃) may be directed to the Carbon Dioxide (CO₂) and Hydrogen (H₂) production process 910.

In embodiments, the Sodium Carbonate (Na₂CO₃) and the Sodium Bicarbonate (NaHCO₃) may be combined with Formic Acid (HCOOH) in the Sodium Formate (HCOONa) production process 916. The Sodium Formate (HCOONa) production process 916 may produce Carbon Dioxide (CO₂) and Sodium Formate (HCOONa). In embodiments, the Sodium Formate (HCOONa) may be directed to the Sodium Formate (HCOONa) decomposition process 917. In embodiments, the Sodium Formate (HCOONa) may be decomposed either thermally or hydrothermally, which may produce Hydrogen (H₂) that may be stored or sent to an EIM to assist with energy production. In embodiments, the Carbon Dioxide (CO₂) produced in the Sodium Formate (HCOONa) production process 916 may be directed to the fuel/cathode side 918 of the SOEC stack 912. The SOEC stack 912 may include the Oxygen (O₂)/Anode side 920. In embodiments, the Oxygen (O₂)/Anode side 920 may be configured to receive purge gas (e.g., Oxygen (O₂), air, Nitrogen (N₂), etc.). The SOEC stack 912 may be configured to produce Oxygen (O₂) for a variety of uses (e.g., hospitals, homes, other industries, etc.). The SOEC stack 912 may be configured to produce Carbon Monoxide (CO) and Carbon Dioxide (CO₂) that may be directed to the Methanol (CH₃OH) production process 914.

For example, a nuclear reactor system may provide electricity, thermal, and/or steam to support reverse osmosis desalination plant where brine treatment is provided, and a chlor-alkali membrane process is conducted in a separate plant. The electricity, thermal, and steam from the nuclear reactor system may also be provided to the chlor-alkali membrane process. From the chlor-alkali membrane process, Chlorine (Cl₂) and Hydrogen (H₂) gas are produced. Resulting Sodium Hydroxide (NaOH) from the chlor-alkali membrane process and clean water from desalination can be fed into DAC system. The NaOH solution containing captured carbon dioxide is processed into Sodium Carbonate (Na₂CO₃) and Sodium Bicarbonate (NaHCO₃). Sodium Formate (HCOONa) may be then applied to a pathway for thermal decomposition described above.

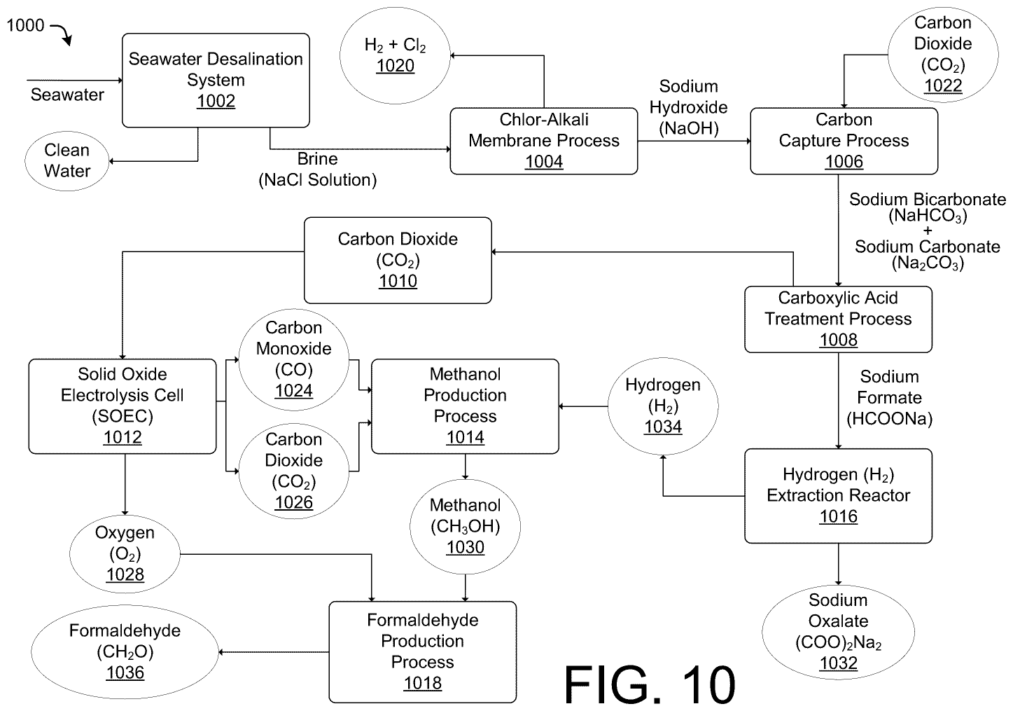

FIG. 10 illustrates a flow diagram of an example process 1000 associated with utilizing a Hydrogen (H₂) extraction reactor (e.g., the Hydrogen (H₂) extraction reactor 1016) and an electrochemical device (e.g., the SOEC 1012) for the simultaneous production of Sodium Oxalate ((COO)₂Na₂) and Formaldehyde (CH₂O). The example process 1000 may include the seawater desalination system 1002, the chlor-alkali membrane process 1004, the carbon capture process 1006, the carboxylic acid process 1008, the SOEC 1012, the Methanol production process 1014, the Hydrogen (H₂) extraction reactor 1016, and the Formaldehyde production process 1018. The process 1000 may include a closed-loop IES for the capturing and production of CO₂ and CO via an NaOH solution, reverse osmosis, and brine treatment for Methanol (CH₃OH) and subsequent Formaldehyde (CH₂O) productions.

In embodiments, the seawater desalination system 1002 may receive seawater and produce clean water and brine (e.g., a concentrated NaCl solution). The brine may be directed into the chlor-alkali membrane process 1004. The chlor-alkali membrane process 1004 may be configured to process the brine to produce Chlorine (Cl₂) and Hydrogen (H₂) 1020, and to regenerate clean water and produce Sodium Hydroxide (NaOH). The Sodium Hydroxide (NaOH) may be directed to the carbon capture process 1006 to be used for carbon capture. In embodiments the carbon capture process 1006 may receive Carbon Dioxide (CO₂) 1022 (e.g., atmospheric air that contains Carbon Dioxide (CO₂)). The carbon capture process 1006 may utilize the Sodium Hydroxide (NaOH) produced by the chlor-alkali membrane process 1004 to capture Carbon Dioxide (CO₂) 1022 to produce Sodium Carbonate (Na₂CO₃) and Sodium Bicarbonate (NaHCO₃). In embodiments, the Sodium Carbonate (Na₂CO₃) and Sodium Bicarbonate (NaHCO₃) may be directed to the carboxylic acid treatment process 1008.

In embodiments, the carboxylic acid treatment process 1008 may include reacting the Sodium Carbonate (Na₂CO₃) and Sodium Bicarbonate (NaHCO₃) with a Carboxylic Acid (R—COOH) (e.g., Formic Acid (HCOOH), etc.) to produce Sodium Formate (HCOONa) and Carbon Dioxide (CO₂) 1010. The Carbon Dioxide (CO₂) 1010 may be directed to the SOEC 1012 to produce two gas streams. One gas stream (e.g., the first gas stream) may include a mixture of Carbon Monoxide (CO) 2024 and Carbon Dioxide (CO₂) 1026. The other gas stream (e.g., the second gas stream) may contain Oxygen (O₂) 1028. In embodiments, the Carbon Monoxide (CO) 1024 and the Carbon Dioxide (CO₂) 1026 may be used in the Methanol production process 1014 to produce Methanol (CH₃OH) 1030. The Hydrogen (H₂) extraction reactor 1016 may receive Sodium Formate (HCOONa) and produce Sodium Oxalate ((COO)₂Na₂) 1032 and Hydrogen (H₂) 1034. For example, the Sodium Formate (HCOONa) may be thermally decomposed to generate Sodium Oxalate ((COO)₂Na₂) 1032 and Hydrogen (H₂) 1034. In embodiments, the Hydrogen (H₂) 1034 may be directed to the Methanol production process 1014 to produce Methanol (CH₃OH) 1030. For example, the Hydrogen (H₂) may be used to react with the Carbon Monoxide (CO) to produce Methanol (CH₃OH) 1030.

In embodiments, the Methanol (CH₃OH) 1030 produced by the Methanol production process 1014 and the Oxygen (O₂) 1028 produced by the SOEC 1012 may be directed to the Formaldehyde production process 1018 to produce Formaldehyde (CH₂O) 1036. For example, Formaldehyde (CH₂O) 1036 may be produced by reacting the Methanol (CH₃OH) 1030 with the Oxygen (O₂) 1028 using moderate reaction temperatures.

For example, seawater undergoes the desalination process and is treated with brine (e.g., an NaCl solution) to produce Chlorine (Cl₂) and Hydrogen (H₂). In the process, clean water is extracted, and Sodium Hydroxide (NaOH) reacts with Carbon Dioxide (CO₂) (from direct air capture) to produce Sodium Carbonate (Na₂CO₃) and Sodium Bicarbonate (NaHCO₃). Carboxylic Acid (e.g., HCOOH) may react with the Sodium Carbonate (Na₂CO₃) and Sodium Bicarbonate (NaHCO₃) to produce Sodium Formate (HCOONa). Sodium Formate (HCOONa) may be heated to produce Sodium Oxalate ((COO)₂Na₂). The released Hydrogen (H₂) may react with Carbon Monoxide (CO) from an SOEC to produce Methanol (CH₃OH). Formaldehyde (CH₂O) may be produced by the of the reaction Methanol (CH₃OH) catalyzed by metals or metal oxides at moderate reaction temperatures.

In embodiments, Carboxylic Acid (HCOOH) reacts with the Sodium Carbonate (Na₂CO₃) and Sodium Bicarbonate (NaHCO₃) to regenerate Carbon Dioxide (CO₂). The Carbon Dioxide (CO₂) is fed into a SOEC to achieve the electrolysis of Carbon Dioxide (CO₂) by using a solid oxide, or ceramic, electrolyte to produce Carbon Monoxide (CO) and Oxygen (O₂).

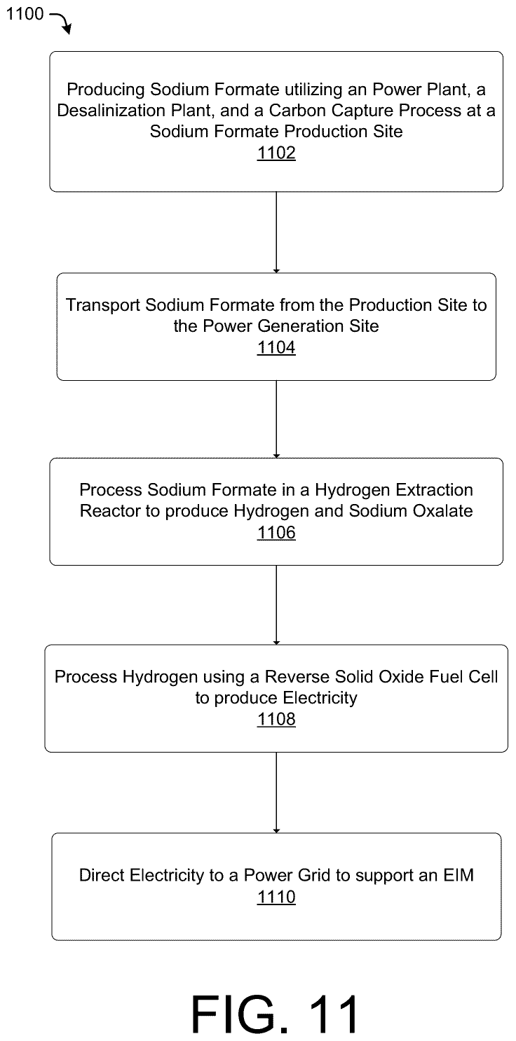

FIG. 11 illustrates a flowchart describing an example process 1100 for utilizing a power plant system to produce Hydrogen (H₂) for use to produce electricity as necessary to support an EIM. The order in which the operations or steps are described is not intended to be construed as a limitation, and any number of the described operations may be combined in any order and/or in parallel to implement the example process 1100.

At step 1102, the example process 1100 may include producing Sodium Formate (HCOONa) at a Sodium Formate (HCOONa) production site by utilizing a power plant, a seawater desalination plant, and a carbon capture process. In embodiments, the power plant may be a SMR system. In embodiments, the desalination plant may be a reverse osmosis desalination plant, a multi-stage flash type distillation plant, or other suitable desalination plant configured to produce clean water and brine from seawater. In embodiments, the carbon capture process may include a Direct Air Capture process. It is understood that the Sodium Formate (HCOONa) production site may be located in close relative proximity to the power plant (i.e., at the same site, etc.).

At step 1104, the example process 1100 may include transporting Sodium Formate (HCOONa) from the Sodium Formate Production Site to the Power Generation Site. In embodiments, the Power Generation Site may include multiple power generation sites. It is understood that the power generation site may be at a location several miles from the Sodium Formate Production Site.

At step 1106, the example process 1100 may include processing the Sodium Formate (HCOONa) transported to the Power Generation Site from the Sodium Formate Production Site in a Hydrogen (H₂) extraction reactor to produce Hydrogen (H₂) and Sodium Oxalate ((COO)₂Na₂).

At step 1108, the example process 1100 may include processing the Hydrogen (H₂) produced in the Hydrogen (H₂) extraction reactor using an electrolysis device (e.g., an RSOFC) to produce electricity.

At step 1110, the example process 1100 may include directing the electricity produced by the RSOEC to a power grid during peak times to support an EIM.

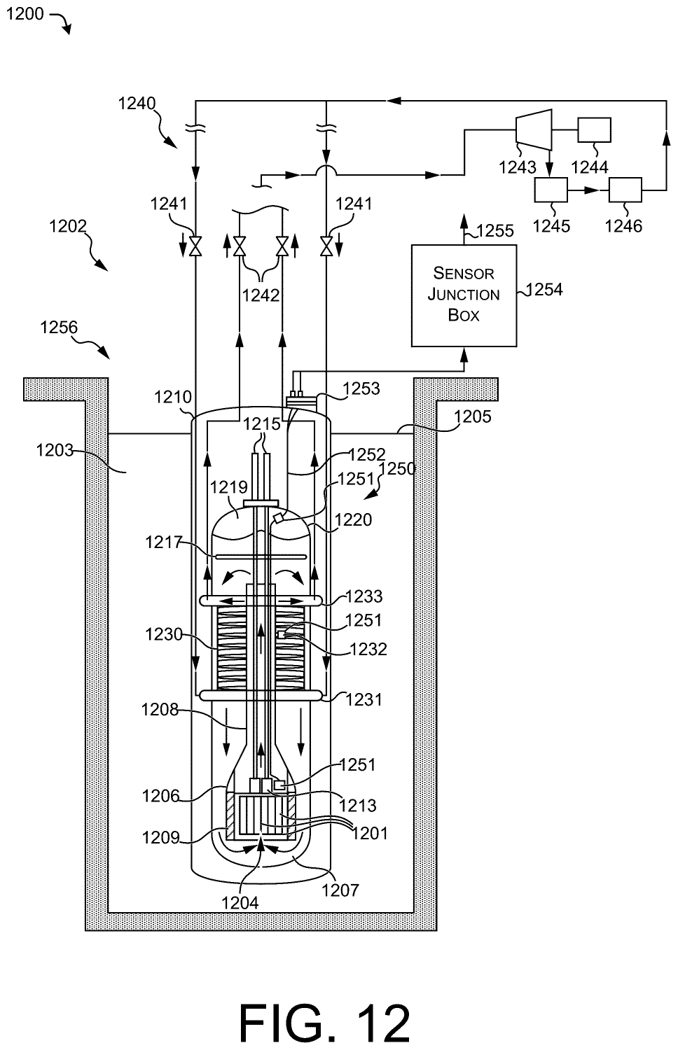

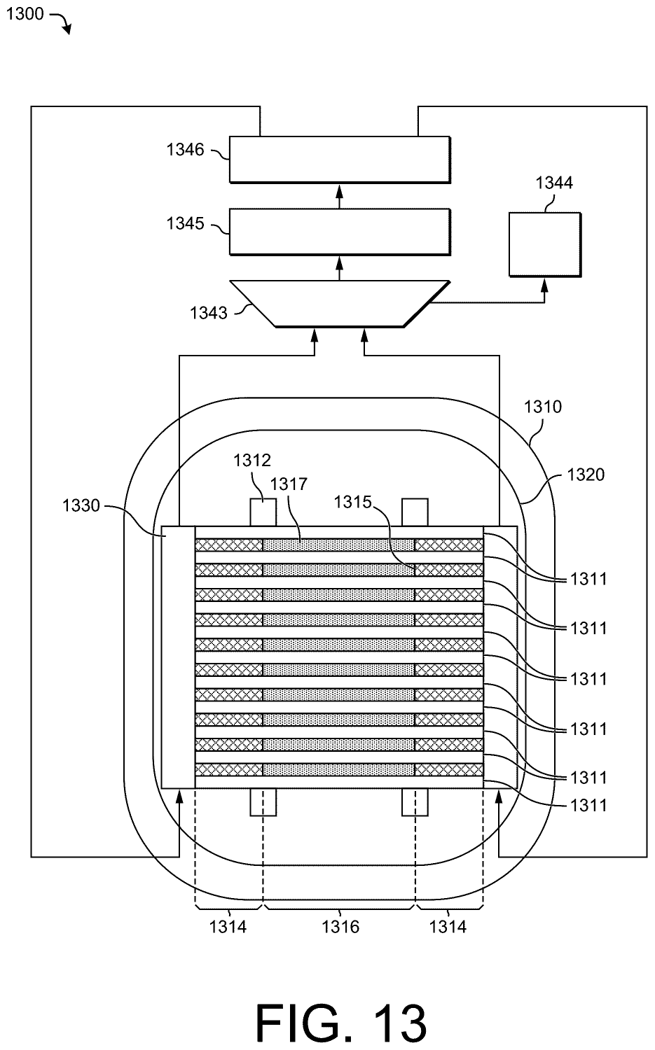

FIGS. 12 and 13 illustrate representative nuclear reactors that may be included in embodiments of the present technology. FIG. 12 is a partially schematic, partially cross-sectional view of a nuclear reactor system 1200 configured in accordance with embodiments of the present technology. The system 1200 can include a power module 1202 having a reactor core 1204 in which a controlled nuclear reaction takes place. Accordingly, the reactor core 1204 can include one or more fuel assemblies 1201. The one or more fuel assemblies 1201 can include fissile and/or other suitable materials. Heat from the reaction generates steam at a steam generator 1230, which directs the steam to a power conversion system 1240. The power conversion system 1240 generates electrical power, and/or provides other useful outputs, such as super-heated steam. A sensor system 1250 is used to monitor the operation of the power module 1202 and/or other system components. The data obtained from the sensor system 1250 can be used in real time to control the power module 1202, and/or can be used to update the design of the power module 1202 and/or other system components.

The power module 1202 includes a containment vessel 1210 (e.g., a radiation shield vessel, or a radiation shield container) that houses/encloses a reactor vessel 1220 (e.g., a reactor pressure vessel, or a reactor pressure container), which in turn houses the reactor core 1204. The containment vessel 1210 can be housed in a power module bay 1256. The power module bay 1256 can contain a cooling pool 1203 filled with water and/or another suitable cooling liquid. The bulk of the power module 1202 can be positioned below a surface 1205 of the cooling pool 1203. Accordingly, the cooling pool 1203 can operate as a thermal sink, for example, in the event of a system malfunction.

A volume between the reactor vessel 1220 and the containment vessel 1210 can be partially or completely evacuated to reduce heat transfer from the reactor vessel 1220 to the surrounding environment (e.g., to the cooling pool 1203). However, in other embodiments the volume between the reactor vessel 1220 and the containment vessel 1210 can be at least partially filled with a gas and/or a liquid that increases heat transfer between the reactor vessel 1220 and the containment vessel 1210. For example, the volume between the reactor vessel 1220 and the containment vessel 1210 can be at least partially filled (e.g., flooded with the primary coolant 1207) during an emergency operation.

Within the reactor vessel 1220, a primary coolant 1207 conveys heat from the reactor core 1204 to the steam generator 1230. For example, as illustrated by arrows located within the reactor vessel 1220, the primary coolant 1207 is heated at the reactor core 1204 toward the bottom of the reactor vessel 1220. The heated primary coolant 1207 (e.g., water with or without additives) rises from the reactor core 1204 through a core shroud 1206 and to a riser tube 1208. The hot, buoyant primary coolant 1207 continues to rise through the riser tube 1208, then exits the riser tube 1208 and passes downwardly through the steam generator 1230. The steam generator 1230 includes a multitude of conduits 1232 that are arranged circumferentially around the riser tube 1208, for example, in a helical pattern, as is shown schematically in FIG. 12. The descending primary coolant 1207 transfers heat to a secondary coolant (e.g., water) within the conduits 1232, and descends to the bottom of the reactor vessel 1220 where the cycle begins again. The cycle can be driven by the changes in the buoyancy of the primary coolant 1207, thus reducing or eliminating the need for pumps to move the primary coolant 1207.

The steam generator 1230 can include a feedwater header 1231 at which the incoming secondary coolant enters the steam generator conduits 1232. The secondary coolant rises through the conduits 1232, converts to vapor (e.g., steam), and is collected at a steam header 1233. The steam exits the steam header 1233 and is directed to the power conversion system 1240.

The power conversion system 1240 can include one or more steam valves 1242 that regulate the passage of high pressure, high temperature steam from the steam generator 1230 to a steam turbine 1243. The steam turbine 1243 converts the thermal energy of the steam to electricity via a generator 1244. The low-pressure steam exiting the turbine 1243 is condensed at a condenser 1245, and then directed (e.g., via a pump 1246) to one or more feedwater valves 1241. The feedwater valves 1241 control the rate at which the feedwater re-enters the steam generator 1230 via the feedwater header 1231. In other embodiments, the steam from the steam generator 1230 can be routed for direct use in an industrial process, such as a Hydrogen (H₂) and Oxygen (O₂) production plant, a chemical production plant, and/or the like, as described in detail below. Accordingly, steam exiting the steam generator 1230 can bypass the power conversion system 1240.

The power module 1202 includes multiple control systems and associated sensors. For example, the power module 1202 can include a hollow cylindrical reflector 1209 that directs neutrons back into the reactor core 1204 to further the nuclear reaction taking place therein. Control rods 1213 are used to modulate the nuclear reaction and are driven via fuel rod drivers 1215. The pressure within the reactor vessel 1220 can be controlled via a pressurizer plate 1217 (which can also serve to direct the primary coolant 1207 downwardly through the steam generator 1230) by controlling the pressure in a pressurizing volume 1219 positioned above the pressurizer plate 1217.

The sensor system 1250 can include one or more sensors 1251 positioned at a variety of locations within the power module 1202 and/or elsewhere, for example, to identify operating parameter values and/or changes in parameter values. The data collected by the sensor system 1250 can then be used to control the operation of the system 1200, and/or to generate design changes for the system 1200. For sensors positioned within the containment vessel 1210, a sensor link 1252 directs data from the sensors to a flange 1253 (at which the sensor link 1252 exits the containment vessel 1210) and directs data to a sensor junction box 1254. From there, the sensor data can be routed to one or more controllers and/or other data systems via a data bus 1255.

FIG. 13 is a partially schematic, partially cross-sectional view of a nuclear reactor system 1300 configured in accordance with additional embodiments of the present technology. In some embodiments, the nuclear reactor system 1300 (“system 1300”) can include some features that are at least generally similar in structure and function, or identical in structure and function, to the corresponding features of the system 1300 described in detail above with reference to FIG. 13, and can operate in a generally similar or identical manner to the system 1300.

In the illustrated embodiment, the system 1300 includes a reactor vessel 1320 and a containment vessel 1310 surrounding/enclosing the reactor vessel 1320. In some embodiments, the reactor vessel 1320 and the containment vessel 1310 can be roughly cylinder-shaped or capsule-shaped. The system 1300 further includes a plurality of heat pipe layers 1311 within the reactor vessel 1320. In the illustrated embodiment, the heat pipe layers 1311 are spaced apart from and stacked over one another. In some embodiments, the heat pipe layers 1311 can be mounted/secured to a common frame 1312, a portion of the reactor vessel 1320 (e.g., a wall thereof), and/or other suitable structures within the reactor vessel 1320. In other embodiments, the heat pipe layers 1311 can be directly stacked on top of one another such that each of the heat pipe layers 1311 supports and/or is supported by one or more of the other ones of the heat pipe layers 1311.

In the illustrated embodiment, the system 1300 further includes a shield or reflector region 1314 at least partially surrounding a core region 1316. The heat pipe layers 1311 can be circular, rectilinear, polygonal, and/or can have other shapes, such that the core region 1316 has a corresponding three-dimensional shape (e.g., cylindrical, spherical). In some embodiments, the core region 1316 is separated from the reflector region 1314 by a core barrier 1315, such as a metal wall. The core region 1316 can include one or more fuel sources, such as fissile material, for heating the heat pipe layers 1311. The reflector region 1314 can include one or more materials configured to contain/reflect products generated by burning the fuel in the core region 1316 during operation of the system 1300. For example, the reflector region 1314 can include a liquid or solid material configured to reflect neutrons and/or other fission products radially inward toward the core region 1316. In some embodiments, the reflector region 1314 can entirely surround the core region 1316. In other embodiments, the reflector region 1314 may partially surround the core region 1316. In some embodiments, the core region 1316 can include a control material 1317, such as a moderator and/or coolant. The control material 1317 can at least partially surround the heat pipe layers 1311 in the core region 1316 and can transfer heat therebetween.

In the illustrated embodiment, the system 1300 further includes at least one heat exchanger 1330 (e.g., a steam generator) positioned around the heat pipe layers 1311. The heat pipe layers 1311 can extend from the core region 1316 and at least partially into the reflector region 1314 and are thermally coupled to the heat exchanger 1330. In some embodiments, the heat exchanger 1330 can be positioned outside of or partially within the reflector region 1314. The heat pipe layers 1311 provide a heat transfer path from the core region 1316 to the heat exchanger 1330. For example, the heat pipe layers 1311 can each include an array of heat pipes that provide a heat transfer path from the core region 1316 to the heat exchanger 1330. When the system 1300 operates, the fuel in the core region 1316 can heat and vaporize a fluid within the heat pipes in the heat pipe layers 1311, and the fluid can carry the heat to the heat exchanger 1330. The heat pipes in the heat pipe layers 1311 can then return the fluid toward the core region 1316 via wicking, gravity, and/or other means to be heated and vaporized once again.

In some embodiments, the heat exchanger 1330 can be similar to the steam generator 1230 of FIG. 12 and, for example, can include one or more helically-coiled tubes that wrap around the heat pipe layers 1311. The tubes of the heat exchanger 1330 can include or carry a working fluid (e.g., a coolant such as water or another fluid) that carries the heat from the heat pipe layers 1311 out of the reactor vessel 1320 and the containment vessel 1310 for use in generating electricity, steam, and/or the like. For example, in the illustrated embodiment the heat exchanger 1330 is operably coupled to a turbine 1343, a generator 1344, a condenser 1345, and a pump 1346. As the working fluid within the heat exchanger 1330 increases in temperature, the working fluid may begin to boil and vaporize. The vaporized working fluid (e.g., steam) may be used to drive the turbine 1343 to convert the thermal potential energy of the working fluid into electrical energy via the generator 1344. The condenser 1345 can condense the working fluid after it passes through the turbine 1343, and the pump 1346 can direct the working fluid back to the heat exchanger 1330 where it can begin another thermal cycle. In other embodiments, steam from the heat exchanger 1330 can be routed for direct use in an industrial process, such as an enhanced oil recovery operation described in detail below. Accordingly, steam exiting the heat exchanger 1330 can bypass the turbine 1343, the generator 1344, the condenser 1345, the pump 1346, etc.

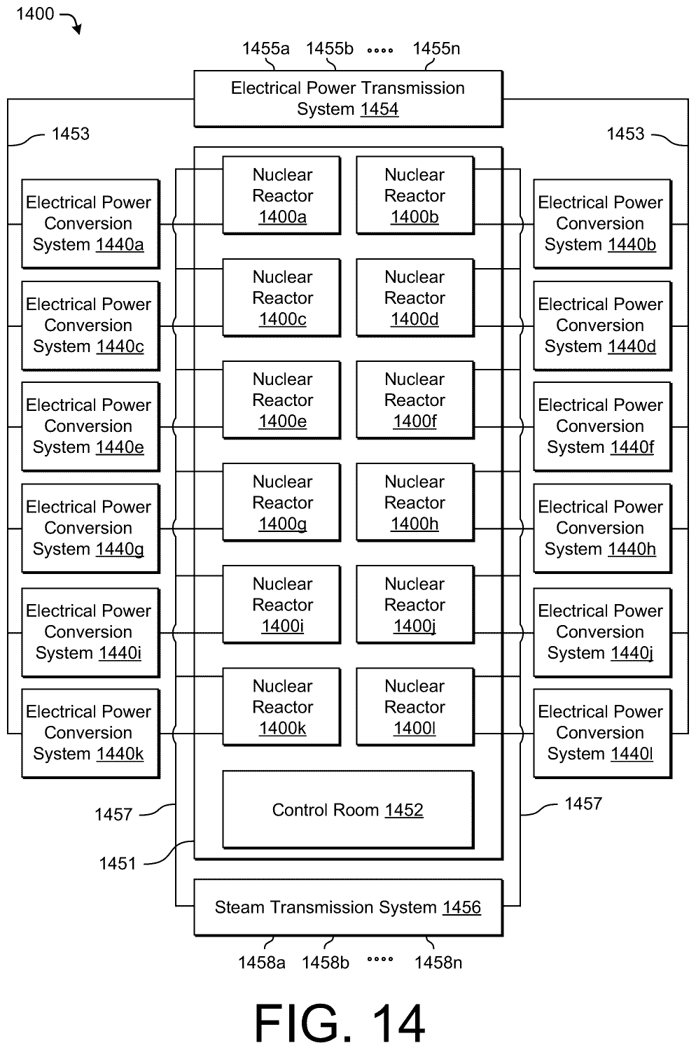

FIG. 14 is a schematic view of a nuclear power plant system 1450 including multiple nuclear reactors 1400 in accordance with embodiments of the present technology. Each of the nuclear reactors 1400 (individually identified as first through twelfth nuclear reactors 1400a-1, respectively) can be similar to or identical to the nuclear reactor 1400 and/or the nuclear reactor 1400 described in detail above with reference to FIGS. 12 and 13. The power plant system 1450 (“power plant system 1450”) can be “modular” in that each of the nuclear reactors 1400 can be operated separately to provide an output, such as electricity or steam. The power plant system 1450 can include fewer than twelve of the nuclear reactors 1400 (e.g., two, three, four, five, six, seven, eight, nine, ten, or eleven of the nuclear reactors 1400), or more than twelve of the nuclear reactors 1400. The power plant system 1450 can be a permanent installation or can be mobile (e.g., mounted on a truck, tractor, mobile platform, and/or the like). In the illustrated embodiment, each of the nuclear reactors 1400 can be positioned within a common housing 1451, such as a reactor plant building, and controlled and/or monitored via a control room 1452.

Each of the nuclear reactors 1400 can be coupled to a corresponding electrical power conversion system 1440 (individually identified as first through twelfth electrical power conversion systems 1440a-1, respectively). The electrical power conversion systems 1440 can include one or more devices that generate electrical power or some other form of usable power from steam generated by the nuclear reactors 1400. In some embodiments, multiple ones of the nuclear reactors 1400 can be coupled to the same one of the electrical power conversion systems 1440 and/or one or more of the nuclear reactors 1400 can be coupled to multiple ones of the electrical power conversion systems 1440 such that there is not a one-to-one correspondence between the nuclear reactors 1400 and the electrical power conversion systems 1440.

The electrical power conversion systems 1440 can be further coupled to an electrical power transmission system 1454 via, for example, an electrical power bus 1453. The electrical power transmission system 1454 and/or the electrical power bus 1453 can include one or more transmission lines, transformers, and/or the like for regulating the current, voltage, and/or other characteristic(s) of the electricity generated by the electrical power conversion systems 1440. The electrical power transmission system 454 can route electricity via a plurality of electrical output paths 1455 (individually identified as electrical output paths 1455a-n) to one or more end users and/or end uses, such as different electrical loads of an integrated energy system.

Each of the nuclear reactors 1400 can further be coupled to a steam transmission system 1456 via, for example, a steam bus 1457. The steam bus 1457 can route steam generated from the nuclear reactors 1400 to the steam transmission system 1456 which in turn can route the steam via a plurality of steam output paths 1458 (individually identified as steam output paths 1458a-n) to one or more end users and/or end uses, such as different steam inputs of an integrated energy system.

In some embodiments, the nuclear reactors 1400 can be individually controlled (e.g., via the control room 1452) to provide steam to the steam transmission system 1456 and/or steam to the corresponding one of the electrical power conversion systems 1440 to provide electricity to the electrical power transmission system 1454. In some embodiments, the nuclear reactors 1400 are configured to provide steam either to the steam bus 1457 or to the corresponding one of the electrical power conversion systems 1440 and can be rapidly and efficiently switched between providing steam to either. Accordingly, in some aspects of the present technology the nuclear reactors 1400 can be modularly and flexibly controlled such that the power plant system 1450 can provide differing levels/amounts of electricity via the electrical power transmission system 1454 and/or steam via the steam transmission system 1456. For example, where the power plant system 1450 is used to provide electricity and steam to one or more industrial process-such as various components of the integrated energy systems, the nuclear reactors 1400 can be controlled to meet the differing electricity and steam requirements of the industrial processes.

As one example, during a first operational state of an integrated energy system employing the power plant system 1450, a first subset of the nuclear reactors 1400 (e.g., the first through sixth nuclear reactors 1400a-f) can be configured to provide steam to the steam transmission system 1456 for use in the first operational state of the integrated energy system, while a second subset of the nuclear reactors 1400 (e.g., the seventh through twelfth nuclear reactors 1400g-1) can be configured to provide steam to the corresponding ones of the electrical power conversion systems 1440 (e.g., the seventh through twelfth electrical power conversion systems 1440g-1) to generate electricity for the first operational state of the integrated energy system. Then, during a second operational state of the integrated energy system when a different (e.g., greater or lesser) amount of steam and/or electricity is required, some or all the first subset of the nuclear reactors 1400 can be switched to provide steam to the corresponding ones of the electrical power conversion systems 1440 (e.g., the seventh through twelfth electrical power conversion systems 1440g-1) and/or some or all of the second subset of the nuclear reactors 1400 can be switched to provide steam to the steam transmission system 1456 to vary the amount of steam and electricity produced to match the requirements/demands of the second operational state. Other variations of steam and electricity generation are possible based on the needs of the integrated energy system. That is, the nuclear reactors 1400 can be dynamically/flexibly controlled during other operational states of an integrated energy system to meet the steam and electricity requirements of the operational state.

In contrast, some conventional nuclear power plant systems can typically generate either steam or electricity for output and cannot be modularly controlled to provide varying levels of steam and electricity for output. Moreover, it is typically difficult (e.g., expensive, time consuming, etc.) to switch between steam generation and electricity generation in conventional nuclear power plant systems. Specifically, for example, it is typically extremely time consuming to switch between steam generation and electricity generation in prototypical large nuclear power plant systems.

The nuclear reactors 1400 can be individually controlled via one or more operators and/or via a computer system. Accordingly, many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described herein. The technology can be embodied in a special-purpose computer, controller or data processor that is specifically programmed, configured, or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described herein may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

### CONCLUSION

During each of the representative production processes described above, Carbon Dioxide (CO₂) generation (e.g., carbon footprints) are either drastically reduce or eliminated. The end results may produce a greener world for our children and grandchildren for many generations to follow.

Although several embodiments have been described in language specific to structural features and/or methodological acts, it is to be understood that the claims are not necessarily limited to the specific features or acts described. Rather, the specific features and acts are disclosed as illustrative forms of implementing the claimed subject matter.

The above detailed description of embodiments of the present technology are not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps may be presented in a given order, in other embodiments, the steps may be performed in a different order. The various embodiments described herein may also be combined to provide further embodiments. Embodiments of the technology disclosed herein may be applied to systems other than those expressly described herein. For example, the technology can be applied to other steam generators with boiling on the inside of tubes, heat exchangers or processing equipment with flow into multiple pipes, and/or similar fluid handling devices that may exhibit density-wave oscillations with potential to cause excessive thermal cycling stresses at the tube or pipe inlets.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. An integrated energy system comprising:
a power plant including at least one nuclear reactor and electrical power generation system, the at least one nuclear reactor being configured to generate steam, and the electrical power generation system being configured to generate electricity;
a desalination system configured to receive at least a portion of the electricity and steam to produce brine;
an electrolysis process configured to process the brine into Sodium Hydroxide (NaOH);
a Sodium Formate (HCOONa) production process configured to receive the Sodium Hydroxide (NaOH) to produce Sodium Formate (HCOONa);
a Hydrogen (H₂) extraction reactor configured to receive the Sodium Formate (HCOONa) and produce Hydrogen (H₂); and
a fuel cell configured to receive the Hydrogen (H₂).

2. The integrated energy system of claim 1, wherein the Hydrogen (H₂) extraction reactor is further configured to produce Sodium Oxalate ((COO)₂Na₂) and includes:
a first rotating spiral configured to direct the Sodium Formate (HCOONa) into an upper portion of the Hydrogen (H₂) extraction reactor, and
a second rotating spiral configured to direct the Sodium Oxalate ((COO)₂Na₂) out of a lower portion of the Hydrogen (H₂) extraction reactor.

3. The integrated energy system of claim 1, wherein:
the Hydrogen (H₂) extraction reactor is further configured to generate at least Carbon Monoxide (CO), and
the Sodium Formate (HCOONa) production process combines the Sodium Hydroxide (NaOH) with the Carbon Monoxide (CO) to produce the Sodium Formate (HCOONa).

4. The integrated energy system of claim 1, further comprising a carbon capture process configured to receive the Sodium Hydroxide (NaOH) to produce a solution containing at least Sodium Hydroxide (NaOH), Sodium Bicarbonate (NaHCO₃), and Sodium Carbonate (Na₂CO₃); and
wherein the Sodium Formate (HCOONa) production process is configured to combine the solution containing at least Sodium Hydroxide (NaOH), Sodium Bicarbonate (NaHCO₃), and Sodium Carbonate (Na₂CO₃) with Formic Acid (HCOOH) to produce Sodium Formate (HCOONa) and Carbon Dioxide (CO₂).

5. The integrated energy system of claim 1, wherein the fuel cell is a Reverse Solid Oxide Fuel Cell (RSOFC), the RSOFC including a thermal recovery system configured to transfer heat from the RSOFC to the Hydrogen (H₂) extraction reactor.

6. The integrated energy system of claim 1, wherein the Hydrogen (H₂) extraction reactor is further configured to produce extracted gases and further comprising a pressure swing adsorption system configured to separate the extracted gases into at least one of Hydrogen (H₂), Carbon Monoxide (CO), or Carbon Dioxide (CO₂).

7. The integrated energy system of claim 1, wherein the Hydrogen (H₂) is collected in a storage tank.

8. A method for electricity production, comprising:
producing steam using a small modular nuclear reactor power plant system;
producing brine utilizing a desalination system configured to receive the steam;
processing the brine utilizing an electrolysis process to produce first Sodium Hydroxide (NaOH);
producing Carbon Monoxide (CO) utilizing a pressure swing adsorption system;
producing first Sodium Formate (HCOONa) by combining the first Sodium Hydroxide (NaOH) with the Carbon Monoxide (CO);
producing a solution including at least second Sodium Hydroxide (NaOH), Sodium Bicarbonate (NaHCO₃), and Sodium Carbonate (Na₂CO₃) utilizing a carbon capture process configured to receive the first Sodium Hydroxide (NaOH);
producing Carbon Dioxide (CO₂) and second Sodium Formate (HCOONa) utilizing the solution and Formic Acid (HCOOH);
producing Hydrogen (H₂) utilizing a Hydrogen (H₂) extraction reactor configured to receive first Sodium Formate (HCOONa) and second Sodium Formate (HCOONa); and
producing electricity utilizing a fuel cell configured to receive the Hydrogen (H₂) to produce electricity.

9. The method of claim 8, wherein the Hydrogen (H₂) extraction reactor is at a temperature within a range of between 360° C.-450° C.

10. The method of claim 8, further comprising:
decomposing the first Sodium Formate (HCOOH) and the second Sodium Formate (HCOOH) into Sodium Oxalate ((COO)₂Na₂) and the Hydrogen (H₂).

11. The method of claim 8, wherein the Carbon Dioxide (CO₂) is first Carbon Dioxide (CO₂), the method further comprising:
receiving the first Carbon Dioxide (CO₂) by a solid oxide electrolysis cell stack to produce second Carbon Dioxide (CO₂) and Carbon Monoxide (CO),
receiving the second Carbon Dioxide (CO₂) and Carbon Monoxide (CO) by the pressure swing adsorption system to produce third Carbon Dioxide (CO₂), and
producing Methanol (CH₃OH) utilizing the third Carbon Dioxide (CO₂).

12. The method of claim 8, wherein the fuel cell includes a thermal recovery system configured to transfer thermal energy generated by the fuel cell to the Hydrogen (H₂) extraction reactor.

13. The method of claim 8, wherein:
the producing the first Sodium Formate (HCOONa) and the producing the second Sodium Formate (HCOONa) takes place at a first site, and
the producing the Hydrogen (H₂) takes place at a second site that is different than the first site.

14. The method of claim 8, further comprising receiving the Hydrogen (H₂) in a storage tank.

15. An integrated system for producing electricity, comprising:
a small modular nuclear reactor power system configured to supply steam;
a desalination system configured to utilize the steam to produce brine;
an electrolysis process configured to produce first Sodium Hydroxide (NaOH) from the brine;
a pressure swing adsorption system configured to produce Carbon Dioxide (CO₂);
a first Sodium Formate (HCOONa) production process configured to produce first Sodium Formate (HCOONa) from the first Sodium Hydroxide (NaOH) and the Carbon Dioxide (CO₂);
a carbon capture process configured to receive the first Sodium Hydroxide (NaOH) and produce a solution including at least second Sodium Hydroxide (NaOH), Sodium Bicarbonate (NaHCO₃), and Sodium Carbonate (Na₂CO₃);
a second Sodium Formate (HCOONa) production process configured to produce second Sodium Formate (HCOONa) from the solution and Formic Acid (HCOOH);
a Hydrogen (H₂) extraction reactor configured to first Sodium Formate (HCOONa) and second Sodium Formate (HCOONa) to produce Hydrogen (H₂); and
a fuel cell configured to utilize the Hydrogen (H₂) to produce electricity.

16. The integrated system of claim 15, wherein the Hydrogen (H₂) extraction reactor includes:
a first rotating spiral configured to direct the first Sodium Formate (HCOONa) and the second Sodium Formate (HCOONa) into an upper portion of the Hydrogen (H₂) extraction reactor, and
a second rotating spiral configured to direct Sodium Oxalate ((COO)₂Na₂) out of a lower portion of the Hydrogen (H₂) extraction reactor.

17. The integrated system of claim 15, further comprising:
a Methanol (CH₃OH) production system configured to receive second Carbon Dioxide (CO₂) to produce Methanol (CH₃OH).

18. The integrated system of claim 15, wherein the fuel cell includes a thermal recovery system configured to transfer thermal energy generated by the fuel cell to the Hydrogen (H₂) extraction reactor.

19. The integrated system of claim 15, wherein:
the first Sodium Formate (HCOONa) production process is at a first site;
the second Sodium Formate (HCOONa) production process is at the first site;
the Hydrogen (H₂) extraction reactor is at a second site that is different than the first site; and
the fuel cell is located at the second site.

20. The integrated system of claim 15, further comprising a storage tank configured to receive the Hydrogen (H₂) from the Hydrogen (H₂) extraction reactor.
